SEQUENCE LISTING (74 sequences)

SEQ ID NO: 1 - DNA, 1410 bp, homo sapiens
  sig_peptide (1)..(57)
atggagtttgtgctgagctgggttttccttgttgctatattaaaaggtgtccagtgtgagggtcagctggtgcaatctgggggaggcttggtacatcctggggggtccctgagactctcctgtgcaggctctggattcaccttcagtagctatggtatgcactgggttcgccaggctccaggaaaaggtctggagtgggtatcaggtattggtactggtggtggcacatactctacagactccgtgaagggccgattcaccatctccagagacaatgccaagaactccttgtatcttcaaatgaacagcctgagagccgaggacatggctgtgtattactgtgcaagaggagattactatggttcggggagtttctttgactgctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 2 - DNA, 711 bp, homo sapiens
  sig_peptide (1)..(66)
atggacatgagggtcctcgctcagctcctggggctcctgctgctctgtttcccaggtgccagatgtgacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctcccacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 3 - DNA, 27 bp, Artificial
  Primer for PCR
gacggatccaccatggagtttgtgctg

SEQ ID NO: 4 - DNA, 27 bp, artificial
  Primer for PCR
gacctcgagtcatttacccggggacag

SEQ ID NO: 5 - DNA, 28 bp, artificial
  Primer for PCR
gacggatccaccatggacatgagggtcc

SEQ ID NO: 6 - DNA, 28 bp, artificial
  Primer for PCR
gacctcgagctaacactctcccctgttg

SEQ ID NO: 7 - DNA, 29 bp, artificial
  Primer for PCR
gacggatccaccatgaaggcgatcctctc

SEQ ID NO: 8 - DNA, 37 bp, artificial
  Primer for PCR
gattgcaccagctgaccctcacgcttcaccaaagggc

SEQ ID NO: 9 - DNA, 37 bp, artificial
  Primer for PCR
gccctttggtgaagcgtgagggtcagctggtgcaatc

SEQ ID NO: 10 - protein, 526 aa, Artificial [sequence omitted]

SEQ ID NO: 11 - DNA, 35 bp, Artificial
  Primer for PCR
ctgggtcatctggatgtcacgcttcaccaaagggc

SEQ ID NO: 12 - DNA, 35 bp, Artificial
  Primer for PCR
gccctttggtgaagcgtgacatccagatgacccag

SEQ ID NO: 13 - protein, 290 aa, Artificial
  Fusion protein of cellulose binding domain from Meripilus giganteus and a kappa light chain from Homo sapiens
MKAILSLAAALLSAAPAFSTAVWGQCGGIGFSGDTTCTASTCVKVNDYYSQCQPGASAPTSTASAPGPSACPLVKRDIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 26 bp, Artificial
  Primer for PCR
gacggatccaccatgaagctactctc

SEQ ID NO: 15 - DNA, 32 bp, Artificial
  Primer for PCR
caccagctgaccctcacgcttcaccaaaggag

SEQ ID NO: 16 - protein, 475 aa, Artificial
  Fusion protein between the prepro sequence from lipase B from Candida antarctica and an IgG1 heavy chain from Homo sapiens where the native signal peptide of the heavy chain has been removed.
MKLLSLTGVAGVLATCVAATPLVKREGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 35 bp, Artificial
  Primer for PCR
ctgggtcatctggatgtcacgcttcaccaaaggag

SEQ ID NO: 18 - DNA, 35 bp, Artificial
  Primer for PCR
ctcctttggtgaagcgtgacatccagatgacccag

SEQ ID NO: 19 - protein, 239 aa, Artificial
  Fusion protein of the prepro sequence from lipase B from Candida antarctica and a kappa light chain from Homo sapiens without its native signal peptide.
MKLLSLTGVAGVLATCVAATPLVKRDIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 27 bp, Artificial
  Primer for PCR
gacggatccaccatggtcgcgtggtgg

SEQ ID NO: 21 - DNA, 32 bp, Artificial
  Primer for PCR
caccagctgaccctcggccaaagcaggtgctg

SEQ ID NO: 22 - protein, 470 aa, Artificial
  Fusion protein of the TAKA signal peptide from Aspergillus oryzae alpha-amylase and a IgG1 heavy chain from Homo sapiens.
MVAWWSLFLYGLQVAAPALAEGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 35 bp, Artificial
  Primer for PCR
ctgggtcatctggatgtcggccaaagcaggtgctg

SEQ ID NO: 24 - DNA, 35 bp, Artificial
  Primer for PCR
cagcacctgctttggccgacatccagatgacccag

SEQ ID NO: 25 - protein, 234 aa, Artificial
  Fusion protein of the TAKA signal peptide sequence from alpha-amylase from Aspergillus oryzae and a kappa light chain from Homo sapiens without its native signal peptide
MVAWWSLFLYGLQVAAPALADIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 20 aa, Aspergillus oryzae
MVAWWSLFLYGLQVAAPALA

SEQ ID NO: 27 - protein, 18 aa, Candida antarctica
MKLLSLTGVAGVLATCVA

SEQ ID NO: 28 - protein, 7 aa, Candida antarctica
ATPLVKR

SEQ ID NO: 29 - DNA, 37 bp, Artificial
  Primer for PCR
gacgacgaattcaagcttatggtgttttgatcatttt

SEQ ID NO: 30 - DNA, 30 bp, Artificial
  Primer for PCR
gacgacgaattcctccaagcatggcatccc

SEQ ID NO: 31 - DNA, 35 bp, Artificial
  Primer for PCR
cctgaattcacgcgcgccaacatgtcttccaagtc

SEQ ID NO: 32 - DNA, 31 bp, Artificial
  Primer for PCR
gttctcgagctacttattgcgcaccaacacg

SEQ ID NO: 33 - DNA, 18 bp, Artificial
  Primer for PCR
accatggcggcactctgc

SEQ ID NO: 34 - DNA, 18 bp, Artificial
  Primer for PCR
gagccgtaggggaagtcc

SEQ ID NO: 35 - DNA, 19 bp, Artificial
  Primer for PCR
cttcagactgaacctcgcc

SEQ ID NO: 36 - DNA, 20 bp, Artificial
  Primer for PCR
gactcggtccgtacattgcc

SEQ ID NO: 37 - DNA, 38 bp, Artificial
  Oligonucleotide for correction of the defect pyrG gene to a functional pyrG gene
cctacggctccgagagaggccttttgatccttgcggag

SEQ ID NO: 38 - DNA, 20 bp, Artificial
  Primer for PCR
gagttagtagttggacatcc

SEQ ID NO: 39 - DNA, 33 bp, Artificial
  Primer for PCR
ctggatgacatgtatcatgaaggtatgtgaatc

SEQ ID NO: 40 - DNA, 32 bp, Artificial
  Primer for PCR
tgttgtctcgagggagagggagaaggagaggg

SEQ ID NO: 41 - DNA, 32 bp, Artificial
  Primer for PCR
cccgcactcgagctgaaatcgacgtggaattc

SEQ ID NO: 42 - DNA, 37 bp, Artificial
  Primer for PCR
gttattttgcggccgccctatccaacgttggacgatc

SEQ ID NO: 43 - DNA, 3374 bp, Aspergillus oryzae
gaattctgtagctgctctatttctattactgtgtattttccttcctctcttaggttgtggaggttgagatagaggttactcttctagatacgggtccaggtccccggtaaataaacgtttcgagggacacaggttagctagtagaatgggaacatctcgatagtatctcagcatgcaagggagagaagttgtacaaaccgcggggcaataagttggcactctaagaatggtgtttctctctcttttggggtcgatttaccgggcgtcggaaacagtgtcgagtgtaaatgcatacatataacactagaatggtagaatgtctaggtatcctgactattatagcttcagatattgccaagtaagaccggaagcgtgactttatatcgcctgacgaaggatagaagagcctacgctaaagcaaagttgttctctcaagtagccctaggtgggaattgctttcgatcgcaaatcaatttgagaaatgccaccaccaccaaatgtaggtcttcaaaactgaaaatcgatcttgattaagcctcgatggatgttcagacttatatatcgttgtagtttgcgtgggatgtacaaccaggtatctacgtcgtggattctgtagggaaagtagtaatgtcttgaggcgactggtaatgtgagtatgcaaggagattgcgagaccggaccacaattactatatcatggtctacccatgatctagcctccgcccagctcactatagatgttcttcagacaggggacggcgaattcacgggcgaaatcaatttgtggctgcatcctcatgtcttgaaacaagcagtgtgattggaagtggtggcgtcttggctccagccagctgcatgactatgagaaaatagtccaactagtcttggcttgcagaatgacttccggaaatttaacctggtggctgctctcagtattggctgacgctccgcacctctaattgtgcttcaggtcagtcaaagatgttttgaaccaccaacgagccgaacccttccgccgctacaatactcgggttattataaaacgcatcgtggttcatcgtcccagctataggattattttcacatcagcaaacgaagtcgaagcaagatggtgtctttctcctcttgtctccgggccttagccctcggatcctcagttctcgcggtccaacctgtccttagacaggcgactggtctagatacctggctgagcacagaagcaaatttttcccgccaggcaatcttgaataatatcggcgcagatggccagtcggcgcagggcgcaagtccaggggtggtgattgccagccctagcaaaagtgacccagattgtacgtgctctggccttttgcaatccttcatttgacttactggccgtagatttctatacctggacccgtgactcgggtctcgtcatgaaaaccctggtcgatctgttcagaggcggagatgccgatcttctccctatcatcgaggagttcattagctcccaggctcggatccaaggcatctcaaacccttctggtgctctttccagtgggggtctgggcgagcctaagttcaatgtcgacgagacagcatttaccggcgcatggggtcggccgcagcgtgacggaccagctttgcgcgcgaccgctatgatctcgtttggagaatggctagttgtaagttccatccgtctttcagaatgggcgtagttaccttataggaaaatagtcatacaagcatagcgacggacctggtatggcctgttgttaggaatgatctatcctatgtagctcagtattggagccaatccgggttcggtgagcttctagagaaatggcccgcagctatgcagtagctaacttccacagatctctgggaggaagtccaaggcacatcattctttactgttgcagtttctcatcgcgctttggtagaaggtagcagcttcgcaaagactgtcggttcctcgtgcccctattgtgactcgcaagcgccccaagtccgatgttatttacaatccttctggaccgggagttacatccaggccaatttcggtggcgggcgatcgggcaaagacatcaacactgtcctgggtagtatccacacgttcgatcctcaagcgacgtgtgatgatgctaccttccagccctgttcggcgcgagcattggccaaccataaggtagtaacggactcgttccgatcaatctatgccatcaactccggtcgtgctgagaatcaagctgttgctgttggccgctaccccgaagacagctattacaacgggaatccttggttcctgaccaccctggccgccgcagagcagttgtatgacgcgttgtaccagtgggataaaattggatcattggccatcacggacgtttctttgccattcttcaaagctctttacagttctgccgcgacagggacctacgcatcgtccacgacggtgtataaggatattgtctcagccgtcaaggcctatgcagacggatacgtgcagatcgtcgtacgtcaagttccctcttcactctcgttaatggttagggttgctaatgaccgcagcaaacctacgctgcatccaccggctccatggccgagcaatataccaagacggacgggagtcagacctccgcccgggatcttacctggtcgtacgctgcacttctcacggccaacaaccgacgaaacgcggtcgttcctgcaccatggggcgagaccgctgccaccagcattccgtcagcttgctctacgacttccgcctcgggcacctacagcagcgtggttatcacatcctggccgaccattagcggatacccaggcgcgccagacagcccctgccaggtgccgacgactgtgtcggtgaccttcgcggtgaaagctactacggtctacggtgagtctatcaagatcgtcgggtcgatctctcagctcgggagctggaatcctagcagcgcgaccgcattgaacgcggacagctacactactgacaaccccttgtggacgggaacaataaacttgcctgctggacagtcgttcgagtataagtttattcgcgttcagaacggggcggttacgtgggagagtgaccccaaccggaaatataccgttccttcgacttgcggggtgaaaagtgctgtgcagagcgatgtttggcggtgatcatcatgtcccgatgaagaggaggaattggtacgtggtattggtgatgtggcgctgtcatattcagtatatatgttcgattctgtgaaatccgaaggcagagagaccaagagagggcccacctattcgtgtgtaatagacaagataatgttagtgtcctatgaaatcccatgatataaatttcgagaagcaggacctacacagatgtatccgattatccttggatcc

SEQ ID NO: 44 - DNA, 29 bp, Artificial
  Primer for PCR
ggaagatctgctgctctatttctattact

SEQ ID NO: 45 - DNA, 30 bp, Artificial
  Primer for PCR
cccaagcttctgggacgatgaaccacgatg

SEQ ID NO: 46 - DNA, 30 bp, Artificial
  Primer for PCR
cccaagcttggatcattggccatcacggac

SEQ ID NO: 47 - DNA, 29 bp, Artificial
  Primer for PCR
cgcgtcgacggatccaaggataatcggat

SEQ ID NO: 48 - DNA, 21 bp, Artificial
  Primer for PCR
gttggccaatgctcgcgccga

SEQ ID NO: 49 - DNA, 21 bp, Artificial
  Primer for PCR
gccctcggatcctcagttctc

SEQ ID NO: 50 - protein, 37 aa, Meripilus giganteus
AVWGQCGGIGFSGDTTCTASTCVKVNDYYSQCQPGAS

SEQ ID NO: 51 - protein, 7 aa, Artificial
  Part of the homo sapiens kappa light chain of ex.3
DIQMTQS

SEQ ID NO: 52 - protein, 8 aa, Artificial
  Part of the homo sapiens gamma heavy chain of ex. 2
EGQLVQSG

SEQ ID NO: 53 - protein, 19 aa, Saccharomyces cerevisiae
  SIGNAL (1)..(19)
MRFPSIFTAVLFAASSALA

SEQ ID NO: 54 - protein, 70 aa, Saccharomyces cerevisiae
  PROPEP (1)..(70)
APVNTTTEDETAQIPAEAVIGYLDLEGDFDVAVLPFSNSTNNGLLFINTTIASIAAKEEGVSLDKREAEA

SEQ ID NO: 55 - DNA, 1344 bp, homo sapiens
gaggtccagctgttggagtctggcggaggcttggtccagcctggggggtccctgaggctctcctgtgccgcctctggtttcacctttagcagctatgccatgagctgggtccgccaggctcctggcaagggcctggaatgggtctccggtattactggcagcggtggtagcacctactacgccgactccgttaagggccgcttcaccatctccagggacaattccaagaacacgctgtatctgcagatgaacagcctgagggccgaggacaccgccgtctattactgcgcgaaagatcccggtactacggttatcatgagctggttcgacccctggggccagggaaccctggtcaccgtctcctccgcctccaccaagggcccttcggtcttccccctggcgccctgctccaggagcacctccgagagcaccgcggccctgggctgcctggtcaaggactacttccccgaacccgttacggtctcgtggaactccggcgctctgaccagcggcgtgcacaccttccccgctgtcctccagtcctccggactctactccctcagcagcgtcgttaccgtgccctccagcaacttcggcacccagacctacacctgcaacgtcgatcacaagcccagcaacaccaaggttgacaagacagttgagcgcaagtgttgcgtcgagtgccctccgtgccccgctccccctgttgctggaccctccgtcttcctcttcccccctaagcccaaggacaccctcatgatctccaggacccctgaggtcacctgcgtcgtggttgacgtcagccacgaagaccccgaggtccagttcaactggtatgtcgacggcgtggaggtgcataacgccaagacaaagccccgtgaggagcagttcaacagcacgttccgtgttgtctccgtcctcaccgttgtgcaccaagactggttgaacggcaaggagtacaagtgcaaggtctccaacaagggcctccccgcccccatcgagaagaccatctccaaaaccaaaggccaaccccgcgaaccccaagtctacaccctgcccccttcccgtgaggagatgaccaagaaccaagtcagcctgacctgcctggtcaagggcttctaccccagcgacatcgccgttgagtgggagagcaatggtcagccggagaacaactacaagaccacccctcccatgctggactccgacggctccttcttcctctactccaaactcaccgtcgacaagagcaggtggcagcagggcaacgtcttctcctgctccgtcatgcatgaggctctgcacaaccactacacgcaaaagagcctctccctgtctccgggcaag

SEQ ID NO: 56 - DNA, 645 bp, homo sapiens
gagatcgtcttgacgcagtctcccggcaccctgtctttgtctcctggcgagagggccaccctctcttgcagggccagccagagcgttcgcggccgctacttggcctggtaccagcagaagcctggccaggctcccaggctcctcatctatggtgcctccagcagggccactggcatccctgacaggttcagcggcagcggttctggcaccgacttcactctcaccatcagcaggctggagcctgaggattttgctgtcttttactgccagcaatatggcagctcgcctcggacgttcggccagggcaccaaggtggaaatcaagcgcactgtggctgctccctctgtcttcatcttcccgccctctgatgagcagttgaagtctggcactgcctctgttgtctgcctgctgaacaacttctatcccagggaggccaaggtccagtggaaggtcgataacgccctccagtcgggcaactcccaggagagcgtcactgagcaggacagcaaggacagcacctacagcctcagcagcaccctgacgctgagcaaggctgactacgagaagcacaaggtctacgcctgcgaagtcacccatcagggcctgagctcgcccgtcaccaagagcttcaacaggggcgagtgc

SEQ ID NO: 57 - DNA, 1572 bp, Artificial
  Fusion of CBD from M.giganteus and IgG2 heavy chain from homo sapiens
atgaaggcgatcctctctctcgctgccgcgctcttgagcgccgcgcccgcgttctcgaccgcagtctggggccagtgcggtggcatcggcttctccggcgacaccacgtgcaccgcttccacctgcgtgaaggtcaacgactactactcgcagtgccagcctggcgcgtccgcacccacgtcaaccgcctccgcgcccggcccgtccgcgtgccctttggtgaagcgtgaggtccagctgttggagtctggcggaggcttggtccagcctggggggtccctgaggctctcctgtgccgcctctggtttcacctttagcagctatgccatgagctgggtccgccaggctcctggcaagggcctggaatgggtctccggtattactggcagcggtggtagcacctactacgccgactccgttaagggccgcttcaccatctccagggacaattccaagaacacgctgtatctgcagatgaacagcctgagggccgaggacaccgccgtctattactgcgcgaaagatcccggtactacggttatcatgagctggttcgacccctggggccagggaaccctggtcaccgtctcctccgcctccaccaagggcccttcggtcttccccctggcgccctgctccaggagcacctccgagagcaccgcggccctgggctgcctggtcaaggactacttccccgaacccgttacggtctcgtggaactccggcgctctgaccagcggcgtgcacaccttccccgctgtcctccagtcctccggactctactccctcagcagcgtcgttaccgtgccctccagcaacttcggcacccagacctacacctgcaacgtcgatcacaagcccagcaacaccaaggttgacaagacagttgagcgcaagtgttgcgtcgagtgccctccgtgccccgctccccctgttgctggaccctccgtcttcctcttcccccctaagcccaaggacaccctcatgatctccaggacccctgaggtcacctgcgtcgtggttgacgtcagccacgaagaccccgaggtccagttcaactggtatgtcgacggcgtggaggtgcataacgccaagacaaagccccgtgaggagcagttcaacagcacgttccgtgttgtctccgtcctcaccgttgtgcaccaagactggttgaacggcaaggagtacaagtgcaaggtctccaacaagggcctccccgcccccatcgagaagaccatctccaaaaccaaaggccaaccccgcgaaccccaagtctacaccctgcccccttcccgtgaggagatgaccaagaaccaagtcagcctgacctgcctggtcaagggcttctaccccagcgacatcgccgttgagtgggagagcaatggtcagccggagaacaactacaagaccacccctcccatgctggactccgacggctccttcttcctctactccaaactcaccgtcgacaagagcaggtggcagcagggcaacgtcttctcctgctccgtcatgcatgaggctctgcacaaccactacacgcaaaagagcctctccctgtctccgggcaag

SEQ ID NO: 58 - protein, 524 aa, Artificial
  Synthetic Construct
MKAILSLAAALLSAAPAFSTAVWGQCGGIGFSGDTTCTASTCVKVNDYYSQCQPGASAPTSTASAPGPSACPLVKREVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - DNA, 873 bp, artificial
  Fusion of CBD from M.giganteus and kappa light from homo sapiens
atgaaggcgatcctctctctcgctgccgcgctcttgagcgccgcgcccgcgttctcgaccgcagtctggggccagtgcggtggcatcggcttctccggcgacaccacgtgcaccgcttccacctgcgtgaaggtcaacgactactactcgcagtgccagcctggcgcgtccgcacccacgtcaaccgcctccgcgcccggcccgtccgcgtgccctttggtgaagcgtgagatcgtcttgacgcagtctcccggcaccctgtctttgtctcctggcgagagggccaccctctcttgcagggccagccagagcgttcgcggccgctacttggcctggtaccagcagaagcctggccaggctcccaggctcctcatctatggtgcctccagcagggccactggcatccctgacaggttcagcggcagcggttctggcaccgacttcactctcaccatcagcaggctggagcctgaggattttgctgtcttttactgccagcaatatggcagctcgcctcggacgttcggccagggcaccaaggtggaaatcaagcgcactgtggctgctccctctgtcttcatcttcccgccctctgatgagcagttgaagtctggcactgcctctgttgtctgcctgctgaacaacttctatcccagggaggccaaggtccagtggaaggtcgataacgccctccagtcgggcaactcccaggagagcgtcactgagcaggacagcaaggacagcacctacagcctcagcagcaccctgacgctgagcaaggctgactacgagaagcacaaggtctacgcctgcgaagtcacccatcagggcctgagctcgcccgtcaccaagagcttcaacaggggcgagtgc

SEQ ID NO: 60 - protein, 291 aa, artificial [sequence omitted]

SEQ ID NO: 61 - DNA, 1404 bp, artificial
  Fusion between TAKA signal peptide and IgG2 heavy chain from homo sapiens
atggtcgcgtggtggtctctatttctgtacggtcttcaggtcgcagcacctgctttggccgaggtccagctgttggagtctggcggaggcttggtccagcctggggggtccctgaggctctcctgtgccgcctctggtttcacctttagcagctatgccatgagctgggtccgccaggctcctggcaagggcctggaatgggtctccggtattactggcagcggtggtagcacctactacgccgactccgttaagggccgcttcaccatctccagggacaattccaagaacacgctgtatctgcagatgaacagcctgagggccgaggacaccgccgtctattactgcgcgaaagatcccggtactacggttatcatgagctggttcgacccctggggccagggaaccctggtcaccgtctcctccgcctccaccaagggcccttcggtcttccccctggcgccctgctccaggagcacctccgagagcaccgcggccctgggctgcctggtcaaggactacttccccgaacccgttacggtctcgtggaactccggcgctctgaccagcggcgtgcacaccttccccgctgtcctccagtcctccggactctactccctcagcagcgtcgttaccgtgccctccagcaacttcggcacccagacctacacctgcaacgtcgatcacaagcccagcaacaccaaggttgacaagacagttgagcgcaagtgttgcgtcgagtgccctccgtgccccgctccccctgttgctggaccctccgtcttcctcttcccccctaagcccaaggacaccctcatgatctccaggacccctgaggtcacctgcgtcgtggttgacgtcagccacgaagaccccgaggtccagttcaactggtatgtcgacggcgtggaggtgcataacgccaagacaaagccccgtgaggagcagttcaacagcacgttccgtgttgtctccgtcctcaccgttgtgcaccaagactggttgaacggcaaggagtacaagtgcaaggtctccaacaagggcctccccgcccccatcgagaagaccatctccaaaaccaaaggccaaccccgcgaaccccaagtctacaccctgcccccttcccgtgaggagatgaccaagaaccaagtcagcctgacctgcctggtcaagggcttctaccccagcgacatcgccgttgagtgggagagcaatggtcagccggagaacaactacaagaccacccctcccatgctggactccgacggctccttcttcctctactccaaactcaccgtcgacaagagcaggtggcagcagggcaacgtcttctcctgctccgtcatgcatgaggctctgcacaaccactacacgcaaaagagcctctccctgtctccgggcaag

SEQ ID NO: 62 - DNA, 705 bp, artificial
  Fusion between the TAKA signal peptide and IgG2 light (kappa) chain from homo sapiens
atggtcgcgtggtggtctctatttctgtacggtcttcaggtcgcagcacctgctttggccgagatcgtcttgacgcagtctcccggcaccctgtctttgtctcctggcgagagggccaccctctcttgcagggccagccagagcgttcgcggccgctacttggcctggtaccagcagaagcctggccaggctcccaggctcctcatctatggtgcctccagcagggccactggcatccctgacaggttcagcggcagcggttctggcaccgacttcactctcaccatcagcaggctggagcctgaggattttgctgtcttttactgccagcaatatggcagctcgcctcggacgttcggccagggcaccaaggtggaaatcaagcgcactgtggctgctccctctgtcttcatcttcccgccctctgatgagcagttgaagtctggcactgcctctgttgtctgcctgctgaacaacttctatcccagggaggccaaggtccagtggaaggtcgataacgccctccagtcgggcaactcccaggagagcgtcactgagcaggacagcaaggacagcacctacagcctcagcagcaccctgacgctgagcaaggctgactacgagaagcacaaggtctacgcctgcgaagtcacccatcagggcctgagctcgcccgtcaccaagagcttcaacaggggcgagtgc

SEQ ID NO: 63 - DNA, 40 bp, artificial
  Primer sequence
gactccaacagctggacctcacgcttcaccaaagggcacg

SEQ ID NO: 64 - DNA, 40 bp, artificial
  Primer sequence
cgtgccctttggtgaagcgtgaggtccagctgttggagtc

SEQ ID NO: 65 - DNA, 39 bp, artificial
  Primer sequence
gactccaacagctggacctcggccaaagcaggtgctgcg

SEQ ID NO: 66 - DNA, 39 bp, artificial
  Primer sequence
cgcagcacctgctttggccgaggtccagctgttggagtc

SEQ ID NO: 67 - DNA, 27 bp, artificial
  Primer sequence
gacctcgagttacttgcccggagacag

SEQ ID NO: 68 - DNA, 40 bp, artificial
  Primer sequence
gactgcgtcaagacgatctcacgcttcaccaaagggcacg

SEQ ID NO: 69 - DNA, 40 bp, artificial
  Primre sequence
cgtgccctttggtgaagcgtgagatcgtcttgacgcagtc

SEQ ID NO: 70 - DNA, 39 bp, artificial
  Primer sequence
gactgcgtcaagacgatctcggccaaagcaggtgctgcg

SEQ ID NO: 71 - DNA, 39 bp, artificial
  Primer sequence
cgcagcacctgctttggccgagatcgtcttgacgcagtc

SEQ ID NO: 72 - DNA, 28 bp, artificial
  Primer sequence
gacctcgagttagcactcgcccctgttg

SEQ ID NO: 73 - protein, 7 aa, artificial
  N-terminal sequence of IgG2 light (kappa) chain
EIVLTQS

SEQ ID NO: 74 - protein, 8 aa, artificial
  N-terminal sequence of IgG2 heavy chain
EVQLLQSG

(19) United States
(12) Patent Application Publication
(10) Pub. No.: US 2006/0024782 A1
(43) Pub. Date: Feb. 2, 2006

(54) Production of a monoclonal antibody in a heterokaryon filamentous fungus or in a filamentous fungal host cell

(75) Inventors: Jan Lehmbeck, Vekso (DK); Fredrik Wahlbom, Malmo (SE)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/041,095

(22) Filed: Jan. 20, 2005

Related U.S. Application Data
(60) Provisional application No. 60/543,227, filed on Feb. 9, 2004.
(60) Provisional application No. 60/580,150, filed on Jun. 16, 2004.

(30) Foreign Application Priority Data
Jan. 21, 2004 (DK) PA 2004 00077
Feb. 5, 2004 (DK) PA 2004 00174
May 12, 2004 (DK) PA 2004 00761

(51) Int. Cl. C07H 21/04; C12P 21/06; C12N 1/14; C12N 1/18; C07K 16/18; C12N 15/74

(52) U.S. Cl. 435/69.1; 435/254.22; 435/254.3; 435/320.1; 530/387.1; 536/23.53

(57) ABSTRACT

The present invention relates to methods for producing a monoclonal antibody in a heterokaryon fungus or in a fungal host cell. Furthermore, it also relates to a nucleic acid construct a first nucleic acid sequence encoding a light chain of an antibody and a third nucleic acid sequence encoding a signal peptide heterologous to the first nucleic acid sequence, a nucleic acid construct comprising a first nucleic acid sequence encoding a heavy chain of an antibody and a third nucleic acid sequence encoding a signal peptide heterologous to the first nucleic acid sequence, a nucleic acid construct comprising a first nucleic acid sequence encoding a light chain of an antibody and a second nucleic acid sequence encoding a cellulose binding domain, and a nucleic acid construct comprising a first nucleic acid sequence encoding a heavy chain of an antibody and a second nucleic acid sequence encoding a cellulose binding domain.

### CROSS-REFERENCE TO RELATED APPLICATIONS

This application claims priority or the benefit under 35 U.S.C. 119 of Danish application nos. PA 2004 00077, PA 2004 00174 and PA 2004 00761 filed Jan. 21, 2004, Feb. 5, 2004 and May 12, 2004, respectively, and U.S. provisional application Ser. Nos. 60/543,227 and 60/580,150 filed Feb. 9, 2004 and Jun. 16, 2004, respectively, the contents of which are fully incorporated herein by reference.

### REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to methods for producing a monoclonal antibody in a heterokaryon fungus or in a fungal host cell.

2. Descripion of Related Art

Monoclonal antibodies have traditionally been expressed in mammalian cells, transgenic animals or plants. However, these systems are less suitable for production on an industrial scale than e.g. bacteria or fungal cell, which have been used extensively for production of different polypeptides on an industrial scale.

U.S. Pat. No. 5,643,745 discloses a heterokaryotic filamentous fungus host capable of producing a heterologous heterodimer comprising at least two subunits.

U.S. Pat. No. 6,331,415 discloses processes for producing an immunoglobin or an immunologically functional immunoglobulin fragment containing at least the variable domains of the immunoglobulin heavy and light chains.

WO 03/089614 discloses methods for the production of monoclonal antibodies in filamentous fungi host cells.

### SUMMARY OF THE INVENTION

The invention provides a method for producing a monoclonal antibody comprising;

(a) providing a heterokaryon fungus comprising a first nucleus and a second nucleus, wherein said first nucleus comprises a first nucleic acid construct comprising a first nucleic acid sequence encoding a light chain of an antibody, and wherein said second nucleus comprises a second nucleic acid construct comprising a first nucleic acid sequence encoding a heavy chain of an antibody, and wherein at least one of said nucleic acid constructs further comprises a second nucleic acid sequence encoding a polypeptide or a functional part thereof normally secreted by a fungus; and

(b) culturing the heterokaryon fungus under conditions suitable for expression of the antibody light and heavy chains.

The present invention also relates to a method for producing a monoclonal antibody comprising;

(a) providing a heterokaryon fungus comprising a first nucleus and a second nucleus, wherein said first nucleus comprises a first nucleic acid construct comprising a first nucleic acid sequence encoding a light chain of an antibody, and wherein said second nucleus comprises a second nucleic acid construct comprising a first nucleic acid sequence encoding a heavy chain of an antibody, and wherein at least one of said nucleic acid constructs further comprises a third nucleic acid sequence encoding a signal peptide heterologous to the first nucleic acid sequence; and

(b) culturing the heterokaryon fungus under conditions suitable for expression of the antibody light and heavy chains.

Furthermore, the present invention also relates to a nucleic acid construct comprising a first nucleic acid sequence encoding a light chain or a heavy chain of an antibody and a third nucleic acid sequence encoding a signal peptide heterologous to the first nucleic acid sequence.

The present invention further relates to a nucleic acid construct comprising a first nucleic acid sequence encoding a light chain or a heavy chain of an antibody and a second nucleic acid sequence encoding a cellulose binding domain.

Moreover, the present invention also relates to heterokaryon fungal host cells.

### BRIEF DESCRIPTION OF THE DRAWINGS

FIG. 1 shows the results of three Western blots of light chain, heavy chain and light+heavy chain expression in a hybridoma cell (Hy) and an Aspergillus oryzae heterokaryon (As). The first gel shows expression of a light chain in a hybridoma cell and an A. oryzae heterokaryon (example 13), the second gel shows expression of a heavy chain in a hybridoma cell and an A. oryzae heterokaryon (example 12) and the third gel shows expression of both light and heavy chain in a hybridoma cell and an A. oryzae heterokaryon (example 14). The first lane of each gel is a standard protein marker used to evaluate the size of the proteins present in the Hy and As lane. The bands observed for the transformant (As) were identified as the heavy chain (50, 53 and 55 kD, probably different glycol forms) and the light chain (25 kD).

FIG. 2 shows the amount of IgG1 antibody expressed by an A. oryzae heterokaryon (described in example 14) as a function of time. The amount of antibody is measured by binding to the antigen and is shown relative to the amount expressed after 168 hours of cultivation of the A. oryzae heterokaryon.

FIG. 3 shows the results of three Western blots of light chain, heavy chain and light+heavy chain expression by an A. oryzae heterokaryon (As). From left to right the first gel shows expression of a heavy chain by an A. oryzae heterokaryon (example 23), the second gel shows expression of both heavy chain an light chain by an A. oryzae heterokaryon (example 23) and the third gel shows expression of the light chain by an A. oryzae heterokaryon (example 23). The first lane of each gel is a standard protein marker used to evaluate the size of the proteins, the second lane is fermentation broth, the third shows the fermentation broth after purification with MepHyperCel and the fourth lane shows the fermentation broth after purification on a ProteinA column.

### DEFINITIONS

The term “heterologous” is in the context of the present invention to be understood as being derived from a different origin, i.e. from cells which are genetically different. Thus the term “heterologous expression” refers to expression of a polypeptide in a host cell, wherein said polypeptide is not expressed by the host cell in nature. The term “a third nucleic acid sequence heterologus to a first nucleic acid sequence” is to be understood as the third and first nucleic acid sequences are derived from different origins, where “origin” may refer to the gene or cell. Thus for example the third and the first nucleic acid sequence may derive from different genes from the same cell or they may derive from similar genes from genetically different cells/species.

The term “homologous” is in the context of the present invention to be understood as being de-rived from the same origin, i.e. from cells which are genetically identical. Thus the term “homologous expression” refers to expression of a polypeptide in a host cell, wherein said polypeptide is expressed by the host cell in nature.

The term “disulfide bond” or “disulfide bridge” is in the context of the present invention to be understood as a covalent bond between the sulphur atoms of two cysteine residues in a polypeptide.

The term “cellulose binding domain” (CBD) is in the context of the present invention to be understood as a polypeptide which binds preferentially to a poly- or oligosaccharide (carbohydrate), frequently—but not necessarily exclusively—to a water-insoluble (including crystalline) form thereof.

CBDs are typically derived from cellulolytic enzymes, i.e. enzymes capable of hydrolyzing cellulose. The CBD is found as an integral part of the polypeptide, which if it is a cellulolytic enzyme further comprises a catalytic domain containing the active site for substrate hydrolysis. Such enzymes may comprise more than one catalytic domain and one, two or three CBDs, and optionally further comprise one or more polypeptide amino acid sequence regions linking the CBD(s) with the catalytic domain(s), a region of the latter type usually being denoted a “linker”. The CBD may be located at the N or C terminus or at an internal position of the polypeptide. That part of the polypeptide constitutes a CBD per se typically consists of more than about 30 and less than about 250 amino acid residues. In particular the CBD of the present invention may be a CBD with the amino acid sequence shown in SEQ ID NO: 50 or an amino acid sequence which has at least 60%, such as at least 70 or 80% homology to the amino acid sequence of SEQ ID NO: 50.

For purposes of the present invention, alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98). Multiple alignments of protein sequences may be made using “ClustalW” (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680). Multiple alignment of DNA sequences may be done using the protein alignment as a template, replacing the amino acids with the corresponding codon from the DNA sequence.

The term “upstream” when used in relation to a nucleic acid sequence or an amino acid sequence is in the context of the present invention to be understood as one or more nucleotide(s) or one or more amino acid(s) physically located on the proximal site of any given point in relation to the 5′−>3′ or N-terminal −>C-terminal direction of the nucleic acid or amino acid sequence, respectively.

The term “downstream” when used in relation to a nucleic acid sequence or an amino acid sequence is in the context of the present invention to be understood as one or more nucleotide(s) or one or more amino acid(s) physically located on the distal site of any given point in relation to the 5′−>3′ or N-terminal −>C-terminal direction of the nucleic acid or amino acid sequence, respectively.

The terms “protein” or “polypeptide” may be used interchangeably in the present invention.

### DETAILED DESCRIPTION OF THE INVENTION

### Monoclonal antibody

The present invention relates to methods for producing a monoclonal antibody in a heterokaryon fungus or in a fungal host cell.

Physiologically antibodies are proteins produced by B-cells (plasma cells) on exposure to an antigen and which possess the ability to react in vitro and in vivo specifically and selectively with the antigenic determinants or epitopes eliciting their production or with an antigenic determinant closely related to the homologous antigen.

In its basic structure antibodies are comprised of two different polypeptide chains; a light chain (approximately 25 kDa) and a heavy chain (approximately 50-70 kDa). Each antibody comprises of a total of four polypeptide chains; two light chains and two heavy chains. In any one antibody the two heavy and the two light chains are identical and the two heavy chains are linked to each other by disulfide bond(s) and each heavy chain is linked to a light chains by a disulfide bond, this gives the antibody its characteristic “Y” shape. The generic term “immunoglobulin” is used for all such proteins. Five different classes of heavy chains have been recognized, i.e. the mu, delta, gamma, alpha and epsilon chains which also defines the class of antibody, i.e. immunoglobulin M (IgM), immunoglobulin D (IgD), immunoglobulin G (IgG), immunoglobulin A (IgA) and immunoglobulin E (IgE), respectively. Furthermore, there are also sub-classes within these five main classes, e.g. in humans four different sub-classes of the gamma type have been recognized, i.e. gamma1, gamma2, gamma3 and gamma4 which produce IgG1, IgG2, IgG3 and IgG4. For the light chains two different types of chains have been recognized; the lambda and the kappa chains.

Both heavy and light chains are divided into distinct structural domains. A mu chain comprises from the N-terminal end a variable region (VH), a first, second, third and fourth constant region (CH1, 2, 3, 4), a delta chain comprises from the N-terminal end a variable region (VH), a first constant region (CH1), a hinge region, a second and a third constant region (CH2, 3), a gamma heavy chain comprises from the N-terminal end a variable region (VH), a first constant region (CH1), a hinge region, a second and third constant region (CH2, 3), an alpha chain comprises from the N-terminal end a variable region (VH), a hinge region, a second and third constant region (CH2, 3) and an epsilon chain comprises from the N-terminal end a variable region (VH), a first, second, third and fourth constant region (CH1, 2, 3, 4).

A light chain comprises a variable region (VL) and a constant region (CL). The different classes of heavy chains differ mainly in the number of constant regions, the presence or absence of a hinge region and the type and/or amount of glycosylation. However, all the different classes of heavy chains comprise a variable region, which is the region capable of binding to/recognizing the antigen.

Generally antibodies are divided into two groups; polyclonal and monoclonal antibodies. Polyclonal antibodies are different (with regard to class and/or subclass of the heavy and/or light chain and/or with regard to the antigenic determinant binding sequences) antibodies which bind to the same antigen. Monoclonal antibodies are identical (with regard to class and subclass of the heavy and light chain, and with regard to the antigenic determinant binding sequences) antibodies. In this context the terms “different” and “identical” refers to the amino acid sequence. Physiologically a monoclonal antibody is synthesized by a single clone of B lymphocytes or plasma cells. The identical copies of the antibody molecules produced contain only one class of heavy chain and one type of light chain. To obtain a homogenous population of antibodies methods for production of monoclonal antibodies have been developed. For example, Kohler and Millstein developed in the mid-1970s B lymphocyte hybridomas by fusing an antibody-producing B lymphocyte with a mutant myeloma cell that was not secreting antibody. Alternatively, antibodies (as Fab fragments or single chains) can be produced and improved by using display systems, e.g. phage display (Rodi, D. et al, 2002. Quantitative assessment of peptide sequence diversity in M13 combinatorial peptide phage display libraries. J. Mol. Biol. 322, 1039-1052).

Different truncated forms of antibodies exist which previously was mainly generated by protease digestion but which today may also be generated by recombinant DNA technology. For example the protease papain cleaves an IgG molecule at the N-terminal side of the disulfide bonds in the hinge region to generate three fragments; two Fab fragments (the arms of the antibody) which each consist of the variable and first constant region of the heavy chain bound to the light chain by a disulfide bond and a Fc fragment which consist of the second and third constant region of both of the heavy chains bound to each other by disulfide bonds at the hinge region.

Another protease pepsin cleaves an IgG molecule at the C-terminal side of the disulfide bonds in the hinge region to generate a F(ab′)2 fragment and a number of small pieces of the Fc fragment. The F(ab′)2 fragment consist of the two Fab fragments from one molecule bound together by disulfide bonds at the hinge region.

Another form of antibodies is the so-called single-chain antibodies, which are antibodies comprising of one light and one heavy chain.

Similar fragments and other fragments of an antibody molecule may be generated by recombinant DNA techniques. Further information about antibodies may be found in e.g. “ImmunoBiology” by Janeway C A and Travers P, Current Biology Ltd./Garland Publishing Inc., 1994 or “Cellular and Molecular Immunology”, Abbas A K, Lichtman A H, Pober J S, W.B. Saunders Publishing, 2003.

Nucleic Acid Construct

The antibody of the present invention is expressed recombinant by a heterokaryon fungus or by a fungal host cell comprising a first and second nucleic acid construct comprising a first nucleic acid sequence encoding a light and a heavy chain, respectively, of an antibody, wherein at least one of said nucleic acid constructs further comprises a second nucleic acid sequence encoding a polypeptide or a functional part thereof normally secreted by a fungus and/or at least one of said nucleic acid constructs further comprises a third nucleic acid sequence encoding a signal peptide heterologous to the first nucleic acid sequence.

In a particular embodiment of the present invention the first nucleic acid construct comprises a first nucleic acid sequence encoding a light chain. In another particular embodiment the first nucleic acid construct may comprise a first nucleic acid sequence encoding a light chain and a second nucleic acid sequence encoding a polypeptide or a functional part thereof normally secreted by a fungus. In another particular embodiment the first nucleic acid construct may comprise a first nucleic acid sequence encoding a light chain and a third nucleic acid sequence encoding a signal peptide heterologous to the first nucleic acid sequence. In another particular embodiment the first nucleic acid construct may comprise a first nucleic acid sequence encoding a light chain and a second nucleic acid sequence encoding a polypeptide or a functional part thereof normally secreted by a fungus and a third nucleic acid sequence encoding a signal peptide heterologous to the first nucleic acid sequence. In particular the first nucleic acid construct may further comprise an inducible promoter, i.e. expression of the first and/or second nucleic acid sequence may be under the control of an inducible promoter. Examples of suitable promoters are given below. In particular the promoter may be obtained from the gene encoding the Aspergillus niger neutral alpha amylase (I or II).

In a particular embodiment of the present invention the second nucleic acid construct comprises a first nucleic acid sequence encoding a heavy chain. In another particular embodiment the second nucleic acid construct may comprise a first nucleic acid sequence encoding a heavy chain and a second nucleic acid sequence encoding a polypeptide or a functional part thereof normally secreted by a fungus. In another particular embodiment the second nucleic acid construct may comprise a first nucleic acid sequence encoding a heavy chain and a third nucleic acid sequence encoding a signal peptide heterologous to the first nucleic acid sequence. In another particular embodiment the second nucleic acid construct may comprise a first nucleic acid sequence encoding a heavy chain and a second nucleic acid sequence encoding a polypeptide or a functional part thereof normally secreted by a fungus and a third nucleic acid sequence encoding a signal peptide heterologous to the first nucleic acid sequence. In particular the second nucleic acid construct may further comprise an inducible promoter, i.e. expression of the first and/or second nucleic acid sequence may be under the control of an inducible promoter. Examples of suitable promoters are given below. In particular the promoter may be obtained from the gene encoding the Aspergillus niger neutral alpha amylase (I or II).

Examples of preferred first, second and third nucleic acid sequences are described below and any combination of these is foreseen.

The present invention also relates to a nucleic acid construct comprising a first nucleic acid sequence encoding a light chain and a third nucleic acid sequence encoding a signal peptide heterologous to the first nucleic acid sequence. In a particular embodiment said signal peptide may be the signal peptide from the alpha-amylase gene from Aspergillus oryzae (the TAKA signal peptide), i.e. the signal peptide depicted in SEQ ID NO: 26, or it may be the signal peptide of the lipase B gene from Candida antarctica, i.e. the signal peptide depicted in SEQ ID NO: 27 or the signal peptide from the mating factor alpha-1 gene from Saccharomyces cerevisiae, i.e. the signal peptide depicted in SEQ ID NO: 53. In particular if the nucleic acid construct comprises the signal peptide of SEQ ID NO: 27 it may further comprise the pro-sequence from the lipase B gene from C. antarctica (SEQ ID NO: 28). In a particular embodiment the nucleic acid construct may further comprise a second nucleic acid sequence encoding a secreted polypeptide or a functional part thereof normally expressed by a fungus, such as a cellulose binding domain (CBD), in particular it may be the CBD shown in SEQ ID NO: 50 or a CBD which has at least 60%, such as 70% or 80% homology to the CBD-sequence of SEQ ID NO: 50.

The present invention also relates to a nucleic acid construct comprising a first nucleic acid sequence encoding a heavy chain and a third nucleic acid sequence encoding a signal peptide heterologous to the first nucleic acid sequence. In a particular embodiment said signal peptide may be the signal peptide of the TAKA amylase gene from Aspergillus oryzae, i.e. the signal peptide depicted in SEQ ID NO: 26, or it may be the signal peptide of the lipase B gene from Candida antarctica, i.e. the signal peptide depicted in SEQ ID NO: 27, or the signal peptide from the mating factor alpha-1 from Saccharomyces cerevisiae, i.e. the signal peptide depicted in SEQ ID NO: 53. In particular if the nucleic acid construct comprises the signal peptide of SEQ ID NO: 27 it may further comprise the pro-sequence from the lipase B gene from C. antarctica (SEQ ID NO: 28). In a particular embodiment the nucleic acid construct may further comprise a second nucleic acid sequence encoding a secreted polypeptide or a functional part thereof normally expressed by a fungus, such as a cellulose binding domain, in particular it may be the CBD shown in SEQ ID NO: 50 or a CBD which has at least 60%, such as 70% or 80% homology to the CBD-sequence of SEQ ID NO: 50.

The present invention also relates to a nucleic acid construct comprising a first nucleic acid sequence encoding a light chain and a second nucleic acid sequence encoding a cellulose binding domain, in particular it may be the CBD shown in SEQ ID NO: 50 or a CBD which has at least 60%, such as 70% or 80% homology to the CBD-sequence of SEQ ID NO: 50.

The present invention also relates to a nucleic acid construct comprising a first nucleic acid sequence encoding a heavy chain and a second nucleic acid sequence encoding a cellulose binding domain, in particular it may be the CBD shown in SEQ ID NO: 50 or a CBD which has at least 60%, such as 70% or 80% homology to the CBD-sequence of SEQ ID NO: 50.

In the following the term “nucleic acid construct” is intended to encompass both first and second nucleic acid constructs.

The terms “first”, “second” and “third” in relation to nucleic acid sequences are not intended to limit the order of said sequences. Particularly, when the nucleic acid construct comprises a first and a third nucleic acid sequence the order of the sequences from the 5′ end may be: 5′-third nucleic acid sequence, first nucleic acid sequence. When the nucleic acid construct comprises a first and second nucleic acid sequence the order of the sequences from the 5′ end may be: 5′-second nucleic acid sequence, first nucleic acid sequence or it may be: 5′-first nucleic acid sequence, second nucleic acid sequence, i.e. the second nucleic acid sequence may be located upstream of the first nucleic acid sequence. When the nucleic acid construct comprises a first, second and third nucleic acid sequence the order of the sequences from the 5′ end may particularly be: 5′-third nucleic acid sequence, second nucleic acid sequence, first nucleic acid sequence or it may be: 5′-third nucleic acid sequence, first nucleic acid sequence, second nucleic acid sequence.

The nucleic acid construct may further comprise other nucleic acid sequences which are relevant for expression of the light or the heavy chain encoded by the first nucleic acid sequence and/or for expression of the polypeptide or functional part thereof normally secreted by a fungus encoded by the second nucleic acid sequence. In particular, the nucleic acid construct may further comprise nucleic acid sequences required or relevant for transcription of the first and/or second nucleic acid sequence or for translation or stability of said transcript or other subsequent processes such as secretion or activation of the polypeptide encode by the first or second nucleic acid sequence. Examples of such further nucleic acid sequences include, but are not limited to, a promoter, a leader, a transcription initiation site, a transcription termination site, a polyadenylation sequence, a propeptide sequence or if the nucleic acid construct does not comprise a third nucleic acid sequence a signal sequence, i.e. a nucleic acid sequence encoding a signal peptide, which may be homologous or heterologous to the first nucleic acid sequence. These further nucleic acid sequences should in particular be “operably linked” to the first and/or second nucleic acid sequence, wherein the term “operably linked” indicates that the further nucleic acid sequences are arranged so that they function in concert for their intended purposes, e.g. transcription initiates in a promoter and proceeds through the first and/or second nucleic acid sequence encoding a polypeptide. In general the further nucleic acid sequences may in particular as a minimum include a promoter, a transcriptional and a translational stop signal.

Promoters

The promoter may be any nucleotide sequence that shows transcriptional activity in the heterokaryon fungus or fungal host cell of choice and may be derived from genes encoding proteins either homologous or heterologous to said heterokaryon or host cell.

In a particular embodiment the promoter may be a so called inducible promoter, i.e. a promoter whose function is determined by the presence or absence of a stimulus, such as the presence or absence of an external compound. Examples of inducible promoters are well known to a person skilled in the art.

Examples of suitable promoters for use in a heterokaryon filamentous fungus or a filamentous fungal host cell include, but are not limited to, promoters obtained from the genes encoding Aspergillus oryzae TAKA amylase, Rhizomucor miehei aspartic proteinase, Aspergillus niger neutral alpha amylase (I or II), Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly, useful promoters include, but are not limited to, the TAKA amylase, NA2-tpi (a hybrid of the promoters from the genes encoding Aspergillus niger neutral (alpha-amylase and Aspergillus oryzae triose phosphate isomerase), and glaA promoters. Further suitable promoters include, but are not limited to, the ADH3 promoter (McKnight et al., The EMBO J. 4 (1985), 2093-2099) or the tpiA promoter.

Examples of suitable promoters for use in a yeast heterokaryon or yeast host cell include but are not limited to promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, N.Y., 1982), or the TPI1 (U.S. Pat. No. 4,599,311) or ADH2-4c (Russell et al., Nature 304 (1983), 652-654) promoters.

Further useful promoters for use in yeast may be obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for use in yeast are described by Romanos et al., 1992, Yeast 8:423488.

Other useful promoter for use in yeast may be obtained from the Pichia pastoris alcohol oxidase (AOX1) gene, the Pichia pastoris glyceraldehydes 3-phosphate dehydrogenase (GAP) gene, and the Pichia pastoris glutathionen-dependent formaldehyde dehydrogenase (FLD1) gene (Cereghino et al., FEMS Microbiology Reviews 24 (2000), 4546).

Transcription Termination Site

A transcription terminator sequence is a sequence recognized by the cell to terminate transcription. The terminator sequence is operably linked downstream of the nucleic acid sequence encoding the polypeptide to be expressed, in the case of the present invention the first and/or second nucleic acid sequence.

Examples of suitable transcription termination sites in particular for use in a filamentous fungus (heterokaryon and/or host cell) include those of, but not limited to, the genes encoding Aspergillus niger neutral alpha-amylase, Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Examples of suitable transcription termination sites in particular for use in a yeast (heterokaryon and/or host cell) include but are not limited to those obtained from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, N.Y., 1982), or the TPI1 (U.S. Pat. No. 4,599,311) or ADH24c (Russell et al., Nature 304 (1983), 652-654) genes.

Further useful transcription termination sites include those obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene.

Other useful transcription termination sites include but are not limited to those obtained from the Pichia pastoris alcohol oxidase (AOX1) gene, the Pichia pastoris glyceraldehydes 3-phosphate dehydrogenase (GAP) gene, and the Pichia pastoris glutathionen-dependent formaldehyde dehydrogenase (FLD1) gene (Cereghino et al., FEMS Microbiology Reviews 24 (2000), 45-46).

Leader Sequence

A leader sequence is a non-translated region of mRNA, which is important for translation by the cell. The leader sequence is operably linked upstream of the nucleic acid sequence encoding the polypeptide to be expressed, in the case of the present invention the first and/or second nucleic acid sequence.

Examples of suitable leader sequences in particular for use in a filamentous fungus (heterokaryon and/or host cell) include those of, but not limited to, the genes encoding Aspergillus oryzae TAKA amylase and Aspergillus oryzae triose phosphate isomerase (TPI) and combinations thereof.

Examples of suitable leader sequences in particular for use in a yeast (heterokaryon and/or host cell) include those obtained from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, N.Y., 1982), or the TPI1 (U.S. Pat. No. 4,599,311) or ADH24c (Russell et al., Nature 304 (1983), 652-654) genes.

Further useful leader sequences in particular for use in yeast (heterokaryon and/or host cell) may be obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene.

Other useful leader sequences in particular for use in yeast (heterokaryon and/or host cell) may be those obtained from the Pichia pastoris alcohol oxidase (AOX1) gene, the Pichia pastoris glyceraldehydes 3-phosphate dehydrogenase (GAP) gene, and the Pichia pastoris glutathionen-dependent formaldehyde dehydrogenase (FLD1) gene (Cereghino et al., FEMS Microbiology Reviews 24 (2000), 45-46).

Polyadenylation Sequences

A polyadenylation sequence is a sequence which when transcribed, is recognized by the cell as a signal to add polyadenosine residues to transcribed mRNA. The polyadenylation sequence is operably linked upstream of the nucleic acid sequence encoding the polypeptide to be expressed, in the case of the present invention the first and/or second nucleic acid sequence.

Examples of suitable polyadenylation sequences in particular for use in a filamentous fungus (heterokaryon and/or host cell) include those of, but not limited to, the genes encoding Aspergillus oryzae TAKA amylase, Aspergillus niger glucoamylase, Aspergillus nidulans anthranilate synthase, and Aspergillus niger alpha-glucosidase.

Examples of suitable polyadenylation sequences in particular for use in a yeast (heterokaryon and/or host cell) include but are not limited to those obtained from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, N.Y., 1982), or the TPI1 (U.S. Pat. No. 4,599,311) or ADH2-4c (Russell et al., Nature 304 (1983), 652-654) genes.

Further useful polyadenylation sequences in particular for use in yeast (heterokaryon and/or host cell) may be those obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene.

Other useful polyadenylation sequences in particular for use in yeast (heterokaryon and/or host cell) include but are not limited to those obtained from the Pichia pastoris alcohol oxidase (AOX1) gene, the Pichia pastoris glyceraldehydes 3-phosphate dehydrogenase (GAP) gene, and the Pichia pastoris glutathionen-dependent formaldehyde dehydrogenase (FLD1) gene (Cereghino et al., FEMS Microbiology Reviews 24 (2000), 45-46).

Propeptide Sequence

A pro-peptide sequence (pro-peptide encoding sequence) is a nucleic acid sequence which encodes an amino acid sequence positioned at the amino terminus of a polypeptide, e.g. in the present invention the polypeptide encoded by the first and/or second nucleic acid sequence. The resultant polypeptide is known as a pro-enzyme or pro-polypeptide (or a zymogen in some cases). A pro-polypeptide is often inactive and can be converted to mature active polypeptide by catalytic or autocatalytic cleavage of the pro-peptide from the pro-polypeptide.

Examples of suitable pro-peptide sequences include those of, but not limited to, the genes encoding Bacillus subtilis alkaline protease (aprE), the Bacillus subtilis neutral protease (nprT), the Saccharomyces cerevisiae mating factor alpha-1 (SEQ ID NO: 54), the Candida antarctica lipase B (SEQ ID NO: 28), Thermomyces lanuginosus lipase or the Myceliophthora thermophilum laccase (WO 95/33836). In particular, if the nucleic acid construct comprises a third nucleic acid sequence encoding the signal peptide from lipase B from C. antarctica (SEQ ID NO: 27) said construct may further comprise the pro-sequence from the same gene (SEQ ID NO: 28).

The first and/or second nucleic acid construct may further comprise a selectable marker, e.g. a gene the product of which complements a defect in the host cell, or a gene encoding resistance to e.g. antibiotics like ampicillin, kanamycin, chloramphenicol, erythromycin, tetracycline, spectinomycine, neomycin, hygromycin, methotrexate, or resistance to heavy metals, virus or herbicides, or which provides for prototrophy or auxotrophs. A suitable selectable marker for use in a filamentous fungus (heterokaryon and/or host cell) may be selected from the group including, but not limited to, amdS (acetamidase), argB (ornithine carbamoyltransferase), bar (phosphinothricin acetyltrans-ferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Particularly, for use in an Aspergillus cell (heterokaryon and/or host cell) are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

Examples of selectable markers suitable for use in yeast (heterokaryon and/or host cell) include but are not limited to ADE2, HIS3, LEU2, LYS2, MET3, TRP1, and URA3.

As described below the heterokaryon fungus is in a particular embodiment obtained by fusion of at least two different strains of fungus wherein the genome of each strain comprises a characteristic which renders the strain dependent on the presence of the genome of the other strain for survival. Thus the first and/or second nucleic acid construct of the heterokaryon fungus may comprise a selectable marker as described above. Said characteristic may also be obtained by co-transformation of one or both of the strains with another nucleic acid construct comprising a selectable marker, as described above.

The purpose of a signal peptide (encoded by a signal sequence also known as a leader sequence, a prepro sequence or a pre sequence) is to direct expression of a polypeptide into the secretory pathway of the cell in which it is expressed. The signal sequence should be joined to the first and/or second nucleic acid sequence in the correct reading frame. Signal sequences are commonly positioned 5′ to the nucleic acid sequence encoding the polypeptide.

Techniques for production of nucleic acid construct, e.g. including ligation, cleavage with restriction enzymes, amplification etc. of the different nucleic acid sequences which are part of the nucleic acid construct are well known to a person skilled in the art and may for example be found in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “Molecular Biological Methods for Bacillus”, John Wiley and Sons, (1990); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “Transcription And Translation”, B. D. Hames & S. J. Higgins, eds. (1984); “Animal Cell Culture”, R. I. Freshney, ed. (1986); “Immobilized Cells And Enzymes”, IRL Press, (1986); “A Practical Guide To Molecular Cloning”, B. Perbal, (1984).

The heterokaryon fungus and/or the fungal host cell of the invention may comprise more than one copy of the first and/or second nucleic acid construct of the present invention to amplify expression of the first nucleic acid sequence and for some embodiments the second nucleic acid sequence. In particular the first and/or second nucleic acid constructs may be integrated in the heterokaryon fungus or the fungal host cell genome. Methods for integration into the genome are well known for a person skilled in the art using methods well known in the art. The nucleic acid construct of the present invention may also comprise one or more nucleic acid sequences which encode one or more factors that are advantageous in the expression of the light and/or heavy and/or the secreted polypeptide or functional part thereof normally expressed by a fungus, e.g., an activator (e.g., a trans-acting factor), a chaperone, or a processing protease. Any factor that is functional in the heterokaryon fungus or the fungal host cell of choice may be used in the present invention. The nucleic acid sequences encoding one or more of these factors may be in tandem with the first and/or second nucleic acid sequence.

First Nucleic Acid Sequence

The first nucleic acid sequence encodes either a light or a heavy chain of an antibody. The first nucleic acid sequences comprised within a particular heterokaryon fungus or fungal host cell of the present invention, should encode a light chain and a heavy chain capable of binding to and/or recognizing the same antigen.

The light chain may be either a kappa or a lambda chain. In particular the light chain may be a kappa chain. The heavy chain may be a mu, delta, gamma, alpha or epsilon chain, in particular it may be a gamma chain. Particularly, the light chain may be a kappa chain and the heavy chain a gamma chain.

The first nucleic acid sequence may derive from any vertebrate organism, in particular it may derive from a human cell.

The first nucleic acid sequence encoding a light chain and the first nucleic acid sequence encoding a heavy chain may derive from different organisms or cells or they may derive from the same organism or cell. In particular both the first nucleic acid sequence encoding a light chain and the first nucleic acid sequence encoding a heavy chain may derive from a human cell.

In a particular embodiment the first nucleic acid sequence may encode a light and heavy chain which binds to an antigen related to or involved in a pathological disease.

Second Nucleic Acid Sequence

The second nucleic acid sequence of the present invention encodes a polypeptide or a functional part thereof normally secreted by a fungus. The three-dimensional structure of many proteins may often be divided into different domains, i.e. spatial areas of the protein. The different domains typically have different functions, e.g. an enzyme may have a domain involved in the enzymatic reaction itself and other domains capable of interacting with other proteins. Often it is possible to express the amino acid sequence of a domain recombinant so that the functionality of the domain is maintained or only slightly changed. Thus in the context of the present invention the term “functional part thereof” is to be understood as a part of the amino acid sequence of a polypeptide normally secreted by a fungus which has the same function as when it is part of said polypeptide. In this context the term “same” means that the function is maintained, e.g. it binds to the same compound as when it is part of the polypeptide, but e.g. the binding kinetics may be altered. For example if the functional part is a domain capable of binding to cellulose expression of the domain by itself without the rest of the polypeptide may result in a domain which is able to bind to cellulose but where e.g. the kinetics relating to said binding may have been changed as compared with the domain when it is part of the polypeptide.

In particular the second nucleic acid sequence of the present invention may encode a cellulose binding domain. In particular it may be the CBD from endoglucanase II derived from Meripilus giganteus, i.e. the sequence shown in SEQ ID NO: 50, or it may be a CBD which has at least 60%, such as 70% or 80% homology to the sequence shown in SEQ ID NO: 50.

Third Nucleic Acid Sequence

The third nucleic acid sequence of the present invention encodes a signal peptide which is heterologous to the first nucleic acid sequence of the invention. A signal peptide is in the context of the present invention to be understood as an amino acid sequence present, often at the N-terminal end, in newly-synthesized forms of secretory and membrane proteins, and which is involved in directing the protein across into the endoplasmic reticulum (ER) in eukaryotic cells. A signal sequence is intended to be understood as a nucleic acid sequence encoding a signal peptide. After translocation of the protein into the ER the signal peptide is usually excised from the protein. Thus in the context of the present invention a signal peptide is to be understood as an amino acid sequence with said function of directing the protein or polypeptide into the ER.

In a particular embodiment the third nucleic acid sequence may be derived from a filamentous fungus, such as Aspergillus, e.g. A. oryzae, A. niger, A. awamori, A. nidulans, A. japonicus, A. phoenicis or A. foetidus, Fusarium, e.g. F. wenenatum, F. oxysporium or F. graminearum, Humicola, e.g. H. insulens or H. lanuginosa, Penicillium, Candida, e.g. C. antarctica, Meripilus, e.g. M. giganteus, or Trichoderma, e.g. T. reesei or T. harzianum.

In particular the third nucleic acid sequence may the sequence depicted in SEQ ID NO: 26, i.e. the TAKA signal peptide derived from the alpha-amylase from Aspergillus oryzae.

In another particular embodiment the third nucleic acid sequence may the sequence depicted in SEQ ID NO: 27, i.e. the signal peptide derived from lipase B from Candida antarctica.

Another example of a third nucleic acid sequence is the signal peptide of the mating factor alpha-1 from Saccharomyces cerevisiae, i.e. the signal peptide depicted in SEQ ID NO: 53.

Heterokaryon Fungus

In one embodiment of the present invention a monoclonal antibody is expressed in a heterokaryon fungus. In the context of the present invention a “heterokaryon” is to be understood as a cell with at least two genetically different nuclei. Heterokaryons derive from fusion of two or more genetically different cells wherein the nuclei of said cells do not fuse resulting in a cell comprising two or more genetically different nuclei.

In particular the heterokaryon fungus may be a filamentous heterokaryon fungus or it may be a yeast heterokaryon. The heterokaryon fungus may be formed naturally between two or more fungi or it may be made artificially. When two or more genetically different fungi fuse the nuclei of each of the individual cells come to coexist in a common cytoplasm. One method to select for heterokaryons is to fuse two or more genetically different cells which each comprise a genome with a characteristic which renders each cell dependent on presence of the nuclei from the other cell for survival. For example if two genetically different cells are fused where each cell type is dependent on a particular nutrient for survival and at the same is independent of the nutrient the other cell depends on then the lack of both nutrients will result in a selection of cells which have fused as here the nuclei from each cells is capable of complementing each other.

The heterokaryon filamentous fungus of the present invention may in particular contain nuclei from cells that are homozygous for all heterokaryon compatibility alleles (except for the mating type allele when the tol gene is present). At least ten chromosomal loci have been identified for heterokaryon incompatibility: het-c, het-d, het-e, het-i, het-5, het-6, het-7, het-8, het-9 and het-10, and more probably exist (see e.g. Perkins et al., “Chromosomal Loci of Neurospora crassa”, Microbiological Reviews (1982) 46: 462-570, at 478).

Formation of the heterokaryon filamentous fungus may in particular be performed by hyphal or protoplast fusion.

In particular the heterokaryon filamentous fungus of the present invention may be made by fusion of hyphae from two different strains of filamentous fungi, wherein the first nuclei of one of the strains contains a genome that results in a characteristic which renders the fungus dependent on the presence of the second nucleus from the other fungus for survival under the conditions provided for fusion to form the heterokaryon, and vice versa. Thus the nucleus of each strain of filamentous fungus confers a characteristic which would result in the failure of the fungus in which it is contained to survive under the culture conditions unless the nucleus from the other filamentous fungus is also present. Examples of characteristics which may be used to render the filamentous fungi strains dependent on each include, but are not limited to, a nutritional requirement, resistance to toxic compounds and resistance to extreme environmental conditions. For example if a first strain which requires the presence of a particular nutrient is cultured on a medium lacking said nutrient along with a second strain which does not require said nutrient for survival, the nucleus of the second strain will confer the ability of a fusion of the two strains to survive even in the absence of the particular nutrient (in this case the second strain and fusions between the first and second strain will survive). Furthermore, if the second strain similarly requires the presence of a particular nutrient different from the nutrient required by the first strain, then only fusions comprising a nucleus from each strain will survive in a medium lacking both of said nutrients.

Methods for formation of a heterokaryon filamentous fungus are described in U.S. Pat. No. 5,643,745 and in the examples of the present invention.

Examples of filamentous fungi which may be fused to form a heterokaryon filamentous fungus include those described below as filamentous fungal host cells. In particular different strains of Aspergillus, e.g. A. oryzae or A. niger, Fusarium or Trichoderma may be used to form a heterokaryon filamentous fungus. In principle, more than two different strains of filamentous fungi may used to form a heterokaryon, such as 3 or 4 or 5 or 6 or 7 or 8 or 9 or 10 different strains. In particular the heterokaryon filamentous fungus of the present invention is formed by fusion of two different strains of filamentous fungi.

Examples of characteristics which make each of the strains of fungi (that are fused to form a heterokaryon filamentous fungus) dependent on the presence of the nucleus from the other fungus for survival under the conditions provided for the fusion include the selectable markers described above. In particular said characteristic may be a characteristic that makes the fungus autotroph. The culture media used for fusion of the different strains of fungi to form a heterokaryon filamentous fungus may be any media which does not complement the particular characteristic of the fungi. Examples of such media are well known to a person skilled in the art as they are generally used to select for recombinant fungi. In the case of fusion of different fungi, however, at least two different characteristics/markers are used for the selection. Examples of characteristics or markers which may be used include those described above as selectable markers useful for the nucleic acid construct. Examples of genes which may make a fungus autotroph include, but are not limited to: pyrG, hemA, niaD, tpi, facC, gala, biA, lysB, sC, methG and phenA. Thus if a fungus is negative for at least one of these genes said gene may be used as a selectable marker.

In Aspergillus nidulans a protein called a Cysteine- and Histidine-Rich-Domain-Containing Protein (CHPA) which is encoded by the cphA gene has been suggested to a have a vital role for maintenance of the diploid phase in A. nidulans. In one embodiment of the present invention the cphA gene or a homolog thereof may have been modified in either the first or second nucleus or both nuclei so that a functional CHPA protein can not be expressed. The modification may be a loss-of-function mutation, such as a null mutation, nonsense or a missense mutation.

In another embodiment the heterokaryon fungus is a yeast heterokaryon. In yeast formation of heterokaryons has been described as a result of a defect in karyogamy (nuclear fusion) during conjugation of two (or more) haploid cells (Olson B L and Siliciano P G, 2003, Yeast, 20, 893-903). A mutation in the kar1 gene in one of the cells to be fused has been described as sufficient to block nuclear fusion during mating (Olson B L and Siliciano P G, Yeast, 2003, 20, 893-903). Thus in a particular embodiment of the present invention at least one of the cells involved in formation of a yeast heterokaryon of the present invention comprises a mutation in the kar1 gene, e.g. a loss-of-function mutation, such as a null mutation, a nonsense or a missense mutation, whereby the nuclei of said cell can not fuse with the nuclei from another cell during mating. The cloning of kar1 in yeast is described in Rose M D and Fink G R, Cell, 1987, 48, 1047-1060.

The nucleic acid construct of the present invention may in particular be introduced into each of the fungi before fusion of said fungi to a heterokaryon fungus. Thus by this method the heterokaryon fungus will comprise at least a first nucleus comprising a first nucleic acid construct and a second nucleus comprising a second nucleic acid construct. Furthermore, as described above the first and second nucleus each comprises a characteristic which makes the fungus dependent on both nuclei. For example the first and/or second nucleus may be negative for at least one of the genes selected from the group consisting of but not limited to: pyrG, hemA, niaD, tpi, facC, gala, biA, lysB, sC, methG and phenA. In particular the first nucleus may be pyrG negative while the second nucleus is hemA negative or vice versa. However, other combinations of characteristics of the two nuclei are foreseen.

Methods of transformation of fungi are well known and may be performed as described below for the fungal host cells. Conditions for culturing a heterokaryon fungus are similar to those for culturing the fungi that it arises from. However, as described above the heterokaryon fungus needs to be cultured in a media selecting for at least two different characteristics. Methods for culturing fungi are well known to a person skilled in the art and may in particular be performed as described further below.

The present invention also relate to a heterokaryon fungal host cell comprising a nucleic acid construct of the present invention.

Furthermore, the present invention also relates to a heterokaryon fungal host cell comprising a first nucleus and a second nucleus, wherein said first nucleus comprises a first nucleic acid construct comprising a first nucleic acid sequence encoding a light chain of an antibody, and wherein said second nucleus comprises a second nucleic acid construct comprising a first nucleic acid sequence encoding a heavy chain of an antibody, and wherein at least one of said nucleic acid constructs further comprises a second nucleic acid sequence encoding a polypeptide or a functional part thereof normally secreted by a fungus.

Furthermore, the present invention also relates to a heterokaryon fungal host cell comprising a first nucleus and a second nucleus, wherein said first nucleus comprises a first nucleic acid construct comprising a first nucleic acid sequence encoding a light chain of an antibody, and wherein said second nucleus comprises a second nucleic acid construct comprising a first nucleic acid sequence encoding a heavy chain of an antibody, and wherein at least one of said nucleic acid constructs further comprises a third nucleic acid sequence encoding a signal peptide heterologous to the first nucleic acid sequence. Preferred embodiments of the first nucleic acid construct, the second nucleic acid construct, the first nucleic acid sequence, the second nucleic acid sequence and the third nucleic acid sequence may be as described above. Different combinations are foreseen, some of which are described below. However, the present invention is not limited to those combinations.

In particular the first nucleus comprises a first nucleic acid construct comprising a first nucleic acid sequence encoding a light chain of an antibody and a second nucleic acid sequence encoding a polypeptide or a functional part thereof normally secreted by a fungus and the second nucleus comprises a second nucleic acid construct comprising a first nucleic acid sequence encoding a heavy chain of an antibody. In another embodiment the first nucleus comprises a first nucleic acid construct comprising a first nucleic acid sequence encoding a light chain of an antibody and the second nucleus comprises a second nucleic acid construct comprising a first nucleic acid sequence encoding a heavy chain of an antibody and a second nucleic acid sequence encoding a polypeptide or a functional part thereof normally secreted by a fungus. In yet another embodiment the first nucleus comprises a first nucleic acid construct comprising a first nucleic acid sequence encoding a light chain of an antibody and a second nucleic acid sequence encoding a polypeptide or a functional part thereof normally secreted by a fungus, and the second nucleus comprises a second nucleic acid construct comprising a first nucleic acid sequence encoding a heavy chain of an antibody and a second nucleic acid sequence encoding a polypeptide or a functional part thereof normally secreted by a fungus. In particular the second nucleic acid sequence may encode a cellulose binding domain. In particular it may be the CBD from endoglucanase II derived from Meripilus giganteus, i.e. the sequence shown in SEQ ID NO: 50, or it may be a CBD which has at least 60%, such as 70% or 80% homology to the sequence shown in SEQ ID NO: 50.

In another embodiment the first nucleus comprises a first nucleic acid construct comprising a first nucleic acid sequence encoding a light chain of an antibody and a third nucleic acid sequence encoding a signal peptide heterologous to the first nucleic acid sequence and the second nucleus comprises a second nucleic acid construct comprising a first nucleic acid sequence encoding a heavy chain of an antibody. In another embodiment the first nucleus comprises a first nucleic acid construct comprising a first nucleic acid sequence encoding a light chain of an antibody and the second nucleus comprises a second nucleic acid construct comprising a first nucleic acid sequence encoding a heavy chain of an antibody and a third nucleic acid sequence encoding a signal peptide heterologous to the first nucleic acid sequence. In yet another embodiment the first nucleus comprises a first nucleic acid construct comprising a first nucleic acid sequence encoding a light chain of an antibody and a third nucleic acid sequence encoding a signal peptide heterologous to the first nucleic acid sequence, and the second nucleus comprises a second nucleic acid construct comprising a first nucleic acid sequence encoding a heavy chain of an antibody and a second nucleic acid sequence encoding a polypeptide or a functional part thereof normally secreted by a fungus. In yet another embodiment the first nucleus comprises a first nucleic acid construct comprising a first nucleic acid sequence encoding a light chain of an antibody and a second nucleic acid sequence encoding a polypeptide or a functional part thereof normally secreted by a fungus, and the second nucleus comprises a second nucleic acid construct comprising a first nucleic acid sequence encoding a heavy chain of an antibody and a third nucleic acid sequence encoding a signal peptide heterologous to the first nucleic acid sequence. In yet another embodiment the first nucleus comprises a first nucleic acid construct comprising a first nucleic acid sequence encoding a light chain of an antibody and a third nucleic acid sequence encoding a signal peptide heterologous to the first nucleic acid sequence, and the second nucleus comprises a second nucleic acid construct comprising a first nucleic acid sequence encoding a heavy chain of an antibody and a third nucleic acid sequence encoding a signal peptide heterologous to the first nucleic acid sequence. In particular the third nucleic acid sequence may the sequence depicted in SEQ ID NO: 26, i.e. the TAKA signal peptide derived from the alpha-amylase from Aspergillus oryzae, or the sequence depicted in SEQ ID NO: 27, i.e. the signal peptide derived from lipase B from Candida antarctica or the mating factor alpha-1 from Saccharomyces cerevisiae, i.e. the signal peptide depicted in SEQ ID NO: 53. The second nucleic acid sequence may in particular encode a cellulose binding domain. In particular it may be the CBD from endoglucanase II derived from Meripilus giganteus, i.e. the sequence shown in SEQ ID NO: 50, or it may be a CBD which has at least 60%, such as 70% or 80% homology to the sequence shown in SEQ ID NO: 50.

Fungi

The second nucleic acid sequence of the present invention derives from a fungus. Moreover, fungi are used both as a host cell and to create a heterokaryon wherein the latter is also used as a host cell in the present invention.

Thus reference to “fungus” or “fungi” in the following is intended to encompass examples of fungi from which the second nucleic acid sequence may derive, fungal host cells of the present invention and fungi which may be used to create a heterokaryon of the present invention. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, In, Ainsworth and Bisby's Dictionary of The Fungi, 8th edition, 1995, CAB International, University Press, Cambridge, UK, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a further embodiment, the fungus of the present invention is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

In a more particular embodiment, the yeast may be a cell of a species of Candida, Kluyveromyces, Saccharomyces, Schizosaccharomyces, Pichia, or Yarrowia. In a most particular embodiment, the yeast may be a Saccharomyces cardsbergensis, Saccharomyces cerevisiae, Saccharomyces diastaticus, Saccharomyces douglasii, Saccharomyces kluyveri, Saccharomyces norbensis or Saccharomyces oviformis cell. In another embodiment, the yeast may be a Kluyveromyces lactis cell. In yet another embodiment, the yeast may be a Yarrowia lipolytica cell.

In another particular embodiment the fungus is a filamentous fungus. Filamentous fungi are characterized by a vegetative mycelium composed of chitin, cellulose, glucan, chitosan, mannan, and other complex polysaccharides. Vegetative growth is by hyphal elongation and carbon catabolism is aerobic.

The filamentous fungus is represented by one of the following groups of Ascomycota: Neurospora, Eupenicillium (=Penicillium), Emericella (=Aspergillus), Eurotium (=Aspergillus).

In a particular embodiment, the filamentous fungus may belong to one of the filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK.). In a more particular embodiment, the filamentous fungus may be a cell of a species of Acremonium, such as A. chrysogenum, or Aspergillus, such as A. awamori, A. foetidus, A. japonicus, A. aculeatus, A. niger or A. oryzae, or Fusarium, such as Fusarium of the section Discolor (also known as the section Fusarium), e.g. F. bactridioides, F. cerealis, F. crookwellense, F. culmorum, F. graminearum (in the perfect state named Gibberella zeae, previously Sphaeria, synonym with Gibberella roseum and Gibberella roseum f.sp. ceralis), F. graminum, F. heterosporum, F. negundi, F. reticulatum, F. roseum, F. sambucinum, F. sarcochroum, F. sulphureum, F. trichothecioides or F. venenatum or a Fusarium strain of the section Elegans, e.g., F. oxysporum, or Humicola, such as H. insolens or H. lanuginose, or Mucor, such as M. miehei, or Myceliophthora, such as M. thermophilum, or Neurospora, such as N. crassa, or Penicillium, such as P. purpurogenum, P. chrysogenum or P. funiculosum (WO 00/68401), or Thielavia, such as T. terrestris, or Tolypocladium, and Trichoderma, such as T. harzianum, T. koningii, T. longibrachiatum, T. reesei or T. viride, or a teleomorph or synonym thereof.

In another particular embodiment of the invention the filamentous fungus may be a protease deficient or protease minus strain. This may for example be the protease deficient strain Aspergillus oryzae JaL 125 having the alkaline protease gene named “alp” deleted. This strain is described in WO 97/35956 (Novozymes), or EP patent no. 429,490, or the TPAP free host cell, in particular a strain of A. niger, disclosed in WO 96/14404. Further, also filamentous fungal cells, especially A. niger or A. oryzae, with reduced production of the transcriptional activator (prtT) as described in WO 01/68864 are specifically contemplated according to the invention. The filamentous fungus may also be toxin and/or mycotoxin free, for instance, free of cyclopiazonic acid, kojic acid, 3-nitropropionic acid and/or aflatoxins. Examples of such strains are disclosed in WO 00/39322 (from Novozymes A/S).

Also filamentous fungi (as described in WO 98/01470) comprising a DNA construct comprising a DNA sequence encoding a transcription factor exhibiting activity in regulating the expression of an alpha-amylase promoter in a filamentous fungus is contemplated according to the invention.

Another example of a suitable filamentous fungus is the JaL355 cell described in example 1 of Danish application no. PA 2003 00169.

The filamentous fungus may be transformed by a process involving protoplast formation, transformation of the protoplasts, and regeneration of the cell wall in a manner known per se. Suitable procedures for transformation of Aspergillus host cells are described in EP 238 023, EP 184,438, and Yelton et al., 1984, Proceedings of the National Academy of Sciences USA 81:1470-1474. A suitable method of transforming Fusarium species is described by Malardier et al., 1989, Gene 78:147-156 or in co-pending U.S. Ser. No. 08/269,449.

Culturing the Fungi

Conditions for culturing fungi which are suitable for expression of protein(s) in said fungus are well-known to a person skilled in the art; see e.g. Spohr, et al. (1998), Journal of Fermentation and Bioengineering, 86 (1), pp. 49-56.

For recombinant expression of a protein the nucleic acid sequence encoding said protein may in particular be under the control of an inducible promoter. In particular if expression of said protein is under control of an inducible promoter culturing of the fungus may be performed in two steps; by first i) culturing the fungus under conditions where the promoter is not induced (non-inducible conditions) and then subsequently ii) under conditions where the promoter is induced (inducible conditions). An advantage of culturing the fungus in such two steps is that during the first step (i) the energy sources are mainly spent on growth of the fungus, while during the second step (ii) the energy sources are mainly spent on production of the recombinant protein. The first step (non-inducible conditions) is also known as the batch phase while the second step (inducible conditions) is also known as the feed phase. During the feed phase fresh medium, known as feed, is typically added to the culture continuously. Different parameters may be used to identify the end of the batch phase and the beginning of the feed phase, such as a change in pH or as described in the examples by the speed of the stirring. Typically, as the fungus grows the dissolved oxygen tension of the fermentation medium reduces and to compensate for this, i.e. to increase it again, the speed of the stirring is often increased. Thus the speed of the stirring may be used as an indirect parameter of the growth of the fungus and hence as when to switch from the batch phase to the feed phase.

In particular the compound used to induce the promoter may also be a compound which the fungus can use as a carbon source, e.g. such as maltose which is described in the examples. However, other promoter inducible systems are well known and may be used in the present invention. For example if the compound used as an inducer is not metabolised by the fungi the amount of inducer and feed rate may be optimized independent of each other.

The inventors of the present invention have found that by slowly and continuously lowering the temperature during the feed phase the amount of antibody expressed by the heterokaryon filamentous fungus is increased. Thus in a preferred embodiment of the present invention culturing the heterokaryon fungus and/or fungal host cell of the present invention may be performed as described above in two steps; a non-inducible phase (batch phase) followed by an inducible phase (feed phase), wherein the temperature is lowered during the inducible phase.

Typically, the temperature during the batch phase is between 28-45° C., such as between 28-40° C., or between 28-38° C., or between 28-36° C., or between 28-34° C., or between 28-32° C., or between 30-40° C., or between 30-38° C., or between 30-36° C., or between 30-34° C., or between 30-32° C.

During the feed phase the temperature may in particular be lowered to a temperature between 16-30° C., such as between 16-28° C., or between 18-28° C., or between 20-28° C., or between 22-28° C., or between 24-28° C., or between 24-26° C., such as approximately 24, 24, 26, 27 or 28° C. In a preferred embodiment the temperature of the feed phase is slowly lowered, e.g. the temperature may be lowered during a period of time corresponding to at least 1/10 of the feed phase, such as ⅕, or ¼, or ⅓, or ½, or ¾ of the feed phase or the temperature may be lowered during the whole feed phase. The temperature may be lowered continuously or step-wise during said period of time.

In particular if the fungus is a filamentous fungi the batch phase typically may take between 10-30 hrs, such as between 12-24 hrs, or between 12-20 hrs, or between 12-18 hrs, or between 12-16 hrs or between 14-24 hrs, or between 14-20 hrs, or between 14-18 hrs, or between 14-16 hrs. The feed phase typically takes more than 50 hrs, such as more than 100 hrs, e.g. between 50-500 hrs, or between 50-250 hrs, or between 50-200 hrs, or between 50-150 hrs, or between 100-250 hrs, or between 100-200 hrs.

However, this depends on the contents of the fermentation of medium and/or the type of filamentous fungus.

Furthermore, the amount of inducer added to the culture and/or the feed rate may also be varied during the feed phase.

Method for Producing a Monoclonal Antibody in a Fungal Host Cell

In another embodiment the present invention relates to a method for producing a monoclonal antibody comprising

(a) providing a fungal host cell comprising a first nucleic acid construct comprising a first nucleic acid sequence encoding a light chain of an antibody and a second nucleic acid construct comprising a first nucleic acid sequence encoding a heavy chain of an antibody, wherein one of said constructs further comprises a second nucleic acid sequence encoding a secreted polypeptide or a functional part thereof normally expressed by a fungus; and

(b) culturing the fungal host cell under conditions suitable for expression of the antibody light and heavy chains.

In particular the fungal host cell may be a filamentous fungal host cell or a yeast host cell, such as one of those described above.

Furthermore, at least one of said nucleic acid constructs may further comprise a third nucleic acid sequence encoding a signal peptide, wherein said signal peptide is heterologous to the first nucleic acid sequence of said nucleic acid construct. In particular the signal peptide may be one of those describe above, e.g. it may be derived from the alpha-amylase gene from Aspergillus oryzae (SEQ ID NO: 26) or the lipase gene from Candida antarctica (SEQ ID NO: 27) or the mating factor alpha-1 gene from Saccharomyces cerevisiae, i.e. the signal peptide depicted in SEQ ID NO: 53.

In particular if the nucleic acid construct comprises the signal peptide of SEQ ID NO: 27 it may further comprise the pro-sequence from the lipase B gene from C. antarctica (SEQ ID NO: 28).

Furthermore, the present invention also relates to a method for producing a monoclonal antibody comprising:

a) providing a fungal host cell comprising a first nucleic acid construct comprising a nucleic acid sequence encoding a light chain of an antibody and a second nucleic acid construct comprising a nucleic acid sequence encoding a heavy chain of an antibody, wherein at least one of said constructs further comprises a third nucleic acid sequence encoding a signal peptide heterologous to the first nucleic acid sequence; and

b) culturing the fungal host cell under conditions suitable for expression of the antibody light and heavy chains.

In particular the fungal host cell may be a filamentous fungal host cell or a yeast host cell, e.g. one of those described above.

Preferred embodiments of the first nucleic acid construct, the second nucleic acid construct, the first nucleic acid sequence, the second nucleic acid sequence and the third nucleic acid sequence may be as described above.

The present invention also relates to a fungal host cell comprising a nucleic acid construct of the present invention.

The present invention also relates to a fungal host cell comprising a first nucleic acid construct comprising a first nucleic acid sequence encoding a light chain of an antibody and a second nucleic acid construct comprising a first nucleic acid sequence encoding a heavy chain of an antibody, wherein one of said constructs further comprises a second nucleic acid sequence encoding a secreted polypeptide or a functional part thereof normally expressed by a fungus.

Furthermore, the present invention also relates to a fungal host cell comprising a first nucleic acid construct comprising a first nucleic acid sequence encoding a light chain of an antibody and a second nucleic acid construct comprising a first nucleic acid sequence encoding a heavy chain of an antibody, wherein at least one of said constructs further comprises a third nucleic acid sequence encoding a signal peptide heterologous to the first nucleic acid sequence. Preferred embodiments of the first and second nucleic acid constructs, the first, second and third nucleic acid sequence may be as described above.

### MATERIALS AND METHODS

### Materials

Strains
Aspergillus oryzae IF04177: available from Institute for fermentation, Osaka; 17-25 Juso Hammachi 2-Chome Yodogawa-Ku, Osaka, Japan.
BECh2 is described in WO 00/39322, example 1, which is further referring to JaL228 described in WO 98/12300, example 1.
JaL355 is described in example 10
ICA133 is described in example 10
ToC1512 is described in example 11

Genes
pyrG: This gene codes for orotidine-5′-phosphate decarboxylase, an enzyme involved in the biosynthesis of uridine.
HemA: This gene codes for delta-aminolevulinate synthase, an enzyme involved in the biosynthesis of heme.

Plasmids
pUC19: The construction is described in Vieira et al, 1982, Gene 19:259-268
pIC19R: is described in Alting-Mees M A and Short J M, 1989, Nucleic Acids Res, 17: 9494
pTAKA-17 is described in EP 0238023
pMT1303 is describe in Uppenberg et al., 1994, Structure 2:293-308
pA2C315 the plasmid is deposited at DSM under the no. DSM971. The plasmid contains a cDNA clone from Meripilus giganteus encoding an endoglucanase II gene.
pJaL676 is described in WO 03/008575, example 5.
pJaL721 is described in WO 03/008575, example 17
pJaL790 is described in example 1
pJaL173 is described in patent WO 98/12300, example 1
pJaL335 is described in patent WO 98/12300, example 1
pDV8 is described in patent WO 01/68864, example 8
pJaL554 is described in patent WO 01/68864, example 8
pToC381 is described in example 11
pToC465 is described in example 11
pToC466 is described in example 11
pICA128 is described in example 10

Primers
Primer H-N (SEQ ID NO: 3)
Primer H-C (SEQ ID NO: 4)
Primer L-N (SEQ ID NO: 5)
Primer L-C (SEQ ID NO: 6)
Primer C315-N (SEQ ID NO: 7)
Primer C315-H-1 (SEQ ID NO: 8)
Primer C315-H-2 (SEQ ID NO: 9)
Primer C315-L-3 (SEQ ID NO: 11)
Primer C315-L-4 (SEQ ID NO: 12)
Primer CalipB-N (SEQ ID NO: 14)
Primer CalipB-H-1 (SEQ ID NO: 15)
Primer CalipB-L-1 (SEQ ID NO: 17)
Primer CalipB-L-2 (SEQ ID NO: 18)
Primer TAKA-17-N (SEQ ID NO: 20)
Primer TAKA-H (SEQ ID NO: 21)
Primer TAKA-L-1 (SEQ ID NO: 23)
Primer TAKA-L-2 (SEQ ID NO: 24)
Primer B6577F12 (SEQ ID NO: 29)
Primer B6575F12 (SEQ ID NO: 30)
Primer 104025 (SEQ ID NO: 31)
Primer 104026 (SEQ ID NO: 32)
Primer 104027 (SEQ ID NO: 33)
Primer 104028 (SEQ ID NO: 34)
Primer 108089 (SEQ ID NO: 35)
Primer 108091 (SEQ ID NO: 36)
Primer 135944 (SEQ ID NO: 38)
Primer B2340E06 (SEQ ID NO: 39)
Primer B2340E07 (SEQ ID NO: 40)
Primer B2340E08 (SEQ ID NO. 41)
Primer B2340E09 (SEQ ID NO: 42)
Primer 101687 (SEQ ID NO: 44)
Primer 101688 (SEQ ID NO: 45)
Primer 101689 (SEQ ID NO: 46)
Primer 101690 (SEQ ID NO: 47)
Primer 101691 (SEQ ID NO: 48)
Primer 101692 (SEQ ID NO: 49)
Primer K1796F02 (SEQ ID NO: 63)
Primer K1796F03 (SEQ ID NO: 64)
Primer K1796F04 (SEQ ID NO: 65)
Primer K1796F05 (SEQ ID NO: 66)
Primer K1795F08 (SEQ ID NO: 67)
Primer K1796F06 (SEQ ID NO: 68)
Primer K1796F07 (SEQ ID NO: 69)
Primer K1796F08 (SEQ ID NO: 70)
Primer K1796F09 (SEQ ID NO: 71)
Primer K1795F09 (SEQ ID NO: 72)

### Methods

General methods of PCR, cloning, ligation of nucleotides etc. are well-known to a person skilled in the art and may for example be found in “Molecular Cloning: A Laboratory Manual”, Sambrook et al. (1989), Cold Spring Harbor Lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

DNA hybridization

In short all DNA hybridizations were carried out for 16 hours at 65° C. in a standard hybridisation buffer of 10× Denhart's solution, 5× SSC, 0.02 M EDTA, 1% SDS, 0.15 mg/ml polyA RNA and 0.05 mg/ml yeast tRNA. After hybridization the filters were washed in 2× SSC, 0.1% SDS at 65° C. twice and exposed to X-ray films.

PCR Amplification

All PCR amplifications were performed in a volume of 100 microL containing 2.5 units Taq po-lymerase, 100 ng of pSO2, 250 nM of each dNTP, and 10 pmol of two of the primers described above in a reaction buffer of 50 mM KCl, 10 mM Tris-HCl pH 8.0, 1.5 mM MgCl₂.

Amplification was carried out in a Perkin-Elmer Cetus DNA Termal 480, and consisted of one cycle of 3 minutes at 94° C., followed by 25 cycles of 1 minute at 94° C., 30 seconds at 55° C., and 1 minute at 72° C.

ELISA for Determination of Intact Human IgG

Intact IgG was determined using an ELISA which uses goat anti-human IgG (Fc specific) as the capture antibody and goat anti-human kappa chain conjugated with alkaline phosphatase as the detection antibody. As standard was used a human myeloma IgG1, kappa purified from human plasma. The ELISA procedure was a standard protocol.

### EXAMPLE 1

Construction of Aspergillus Expression Plasmid pJaL790

The Aspergillus expression plasmid pJaL790 was constructed in the following way:

The single restriction endonuclease site HindIII in the vector pUC19 was removed by cutting with HindIII and the free overhand-ends was filled out by treatment with Klenow polymerase and the four deoxyribonucleotides and ligated, resulting in plasmid pJaL720. The 1140 bp EcoRI-BamHI fragment from pJaL721 was cloned into the corresponding sites in pJaL720, resulting in pJaL723. A 537 bp fragment was amplified by PCR with pJaL676 as template and the primers B6577F12 (SEQ ID NO: 29) and B6575F12 (SEQ ID NO: 30). This was digested with EcoRI, the free overhand-ends was filled out by treatment with Klenow polymerase and the four deoxyribonucleotides and the obtained 524 bp fragment was cloned into the HindIII site, which was blunt ended in pJaL723, giving plasmid pJaL728. The single restriction endonuclease site HindIII in the vector pUC19 was removed by cutting with HindIII and the free overhand-ends was filled out by treatment with Klenow polymerase and the four deoxyribonucleotides and ligated, resulting in plasmid pJaL784. A 1671 bp EcoRI-BamHI fragment from pJaL784 was ligated to the 5735 bp EcoRI-BamHI fragment from pJaL721, resulting in pJaL790.

### EXAMPLE 2

Construction of a Native IgG1 Heavy-chain Asperillus Expression Plasmid

A human IgG1 heavy chain encoding sequence was amplified by PCR using SEQ ID NO: 1 as template and the forward primer H-N (SEQ ID NO: 3) and the reverse primer H-C (SEQ ID NO: 4). Primer H-N and HL-C introduce a BamHI and XhoI restriction site upstream the translational start codon and after the translation termination signal, respectively, for cloning purposes. The PCR product on 1431 bp was purified and cut with the restriction endonucleases BamHI and XhoI. The resulting 1419 bp fragment was cloned into the corresponding site in pJaL790 to create pNZ-3. DNA from clone pNZ-3 was sequenced to check that it was the right sequence.

### EXAMPLE 3

Construction of a Native kappa Light-chain Aspergillus Expression Plasmid

A human kappa light-chain encoding sequence was amplified by PCR using SEQ ID NO: 2 as template and with the forward primer L-N (SEQ ID NO: 5) and the reverse primer L-C (SEQ ID NO: 6). Primer L-N and L-C introduce a BamHI and XhoI restriction site upstream the translational start codon and after the translation termination signal, respectively, for cloning purposes. The PCR product on 732 bp was purified and cut with the restriction endonucleases BamHI and XhoI. The resulting 720 bp fragment was cloned into the corresponding site in pJaL790 to create pNZ4. DNA from clone pNZ4 was sequenced to check that it was the right sequence.

### EXAMPLE 4

Construction of an IaG1 Heavy-chain CBD Fusion Aspergillus Expression Vector

A fusion protein between the human IgG1 heavy chain used in example 2 and a cellulose binding domain from the endoglucanase II from M. giganteus was constructed by exchanging the DNA sequence encoding the native signal peptide of the heavy chain with the DNA sequence encoding the M. giganteus cellulose binding domain having its own signal and a linker ending with amino acids KR (CBD) by sequence overlap extension (SOE). The CBD was amplified by PCR using pA2C315 as template and the following pair of primers: the forward primer C315-N (SEQ ID NO: 7) and the reverse primer C315-H-1 (SEQ ID NO: 8). The resulting PCR product on 260 bp was purified. The primer C315-N introduces a BamHI restriction site upstream of the translation start codon for cloning purposes.

The heavy-chain was amplified by PCR using pNZ-3 as template and the following pair of primers: the forward primer C315-H-2 (SEQ ID NO: 9) and the reverse primer H-N (SEQ ID NO: 3). The resulting PCR product on 1379 bp was purified.

The two above PCR products were mixed and a standard SOE PCR was preformed with the following pair of primers C315-N (SEQ ID NO: 7) and H-N (SEQ ID NO: 3) resulting in an amplified fragment of 1602 bp. The 1602 bp fragment was purified and cut with BamHI and XhoI. The resulting 1590 bp fragment was cloned into the corresponding restriction endonuclease sites of pJaL790 giving an Aspergillus expression plasmid named pNZ-5. The complete amino acid sequence of the CBD fused to the heavy-chain is given in SEQ ID NO: 10.

### EXAMPLE 5

Construction of kappa Light-chain CBD Fusion Aspergillus Expression Vector

A fusion protein between the human kappa light chain used in example 3 and a cellulose binding domain from the endoglucanase II from M. giganteus was constructed by exchanging the DNA sequence encoding the native signal peptide of the light chain with the DNA sequence encoding the M. giganteus cellulose binding domain having its own signal and a linker ending with amino acids KR (CBD) by sequence overlap extension (SOE).

The CBD was amplified by PCR on pA2C315 using the following pair of primers: the forward primer C315-N (SEQ ID NO: 7) and the reverse primer C315-L-3 (SEQ ID NO: 11). The resulting PCR product on 258 bp was purified. The primer C315-N introduces a BamHI restriction site upstream of the translation start codon for cloning purposes.

The light-chain was amplified by PCR out from pNZ-4 using the following pair of primers: the forward primer C315-L-4 (SEQ ID NO: 12) and the reverse primer L-N (SEQ ID NO: 5). The resulting PCR product on 671 bp was purified.

The two above PCR products were mixed and a standard SOE PCR was preformed with the following pair of primers C315-N and L-N resulting in an amplified fragment of 894 bp. The 894 bp fragment was purified and cut with BamHI and XhoI. The resulting 797 bp fragment was cloned into the corresponding restriction endonuclease sites of pJaL790 giving an Aspergillus expression plasmid named pNZ-6. The complete amino acid sequence of the CBD fused to the light-chain is given in SEQ ID NO: 13.

### EXAMPLE 6

Construction of IgG1 Heavy-chain Aspergillus Expression Plasmid, Where the Native Signal Peptide of the Heavy Chain is Exchanged with the Prepro Sequence from Lipase B from Candida antarctica

The signal peptide sequence of the human IgG1 heavy-chain used in example 2 was exchanged with the prepro sequence from lipase B from Candida antarctica by sequence overlap extension (SOE). The prepro region was amplified by PCR with pMT1303 as template and the following pair of primers: the forward primer CalipB-N (SEQ ID NO: 14) and the reverse primer CalipB-H-1 (SEQ ID NO: 15). The resulting PCR product on 105 bp was restriction endonuclease digested with BamHI and PvuII and the 92 bp fragment was purified. The primer CalipB-N introduces a BamHI restriction site upstream of the translation start codon for cloning purposes. pNZ3 was digested with the restriction enzymes PvuII and XhoI and the 1345 bp fragment encoding the heavy-chain was purified.

The two above fragments on 92 bp and on 1435 bp was cloned into pJaL790 digested with restriction endonucleases BamHI and XhoI, resulting in an Aspergillus expression plasmid named pNZ-7. The complete amino acid sequence of the heterologous signal fused to the heavy-chain is given in SEQ ID NO: 16.

### EXAMPLE 7

Construction of kappa Light-chain Aspergillus Expression Plasmid, Where the Native Signal is Exchanged with the Candida Lipase B Prepro

The signal peptide sequence of the human kappa light-chain used in example 3 was exchanged with the prepro sequence from lipase B from Candida antarctica by sequence overlap extension (SOE). The prepro region were amplified by PCR on pMT1303 using the following pair of primers: the forward primer CalipB-N (SEQ ID NO: 14) and the reverse primer CalipB-L-1 (SEQ ID NO: 17). The resulting PCR product on 105 bp was purified. The light-chain was amplified by PCR with pNZ-4 as template using the following pair of primers: the forward primer CalipB-L-2 (SEQ ID NO: 18) and the reverse primer L-N (SEQ ID NO: 5). The resulting PCR product on 671 bp was purified. The primer CalipB-N introduces a BamHI restriction site upstream of the translation start codon for cloning purposes.

The two above PCR products were mixed and a standard SOE PCR was preformed with the following pair of primers CalipB-N (SEQ ID NO: 14) and L-N (SEQ ID NO: 5) resulting in an amplified fragment of 741 bp. The 741 bp fragment was purified and cut with restriction endonucleases BamHI and XhoI. The resulting 729 bp fragment was cloned into the corresponding restriction endonuclease sites of pJaL790 giving an Aspergillus expression plasmid named pNZ-8. The complete amino acid sequence of the heterologous signal fused to the light-chain is given in SEQ ID NO: 19.

### EXAMPLE 8

Construction of IgG1 Heavy-chain Aspergillus Expression Plasmid, Where the Native Signal is Exchanged with the TAKA Signal

The signal peptide sequence of the human IgG1 heavy-chain used in example 2 was exchanged with the signal peptide sequence from Aspergillus oryzae alpha-amylase (TAKA) by sequence overlap extension (SOE). The TAKA signal was amplified by PCR on pTAKA17 using the following pair of primers: the forward primer TAKA-17-N (SEQ ID NO: 20) and the reverse primer TAKA-H (SEQ ID NO: 21). The primer TAKA-17-N introduces a BamHI restriction site upstream of the translation start codon for cloning purposes. The resulting PCR product on 90 bp was restriction digested with BamHI and PvuII and the resulting 80 bp fragment was purified. pNZ3 was restriction digested with PvuII and XhoI and the 1345 bp fragment encoding the heavy-chain was purified.

The two above fragments on 80 bp and on 1435 bp was cloned into pJaL790 digested with restriction endonucleases BamHI and XhoI, resulting in an Aspergillus expression plasmid named pNZ-9. The complete amino acid sequence of the heterologous signal fused to the heavy-chain is given in SEQ ID NO: 22.

### EXAMPLE 9

Construction of kappa Light-chain Aspergillus Expression Plasmid, Where the Native Signal is Exchanged with the TAKA Signal

The signal peptide sequence of the human kappa light-chain used in example 3 was exchanged with the signal peptide sequence from A. oryzae alpha-amylase (TAKA) by sequence overlap extension (SOE). The TAKA signal was amplified by PCR using pTAKA17 as template and the following pair of primers: the forward primer TAKA-17-N (SEQ ID NO: 20) and the reverse primer TAKA-L-1 (SEQ ID NO: 23). The primer TAKA-17-N introduces a BamHI restriction site upstream of the translation start codon for cloning purposes. The resulting PCR product on 93 bp was purified. The light-chain was amplified by PCR on pNZ-4 using the following pair of primers: the forward primer TAKA-L-2 (SEQ ID NO: 24) and the reverse primer L-C (SEQ ID NO: 6). The resulting PCR product on 671 bp was purified.

The two above PCR products were mixed and a standard SOE PCR was preformed with the following pair of primers TAKA-17-N (SEQ ID NO: 20) and L-C (SEQ ID NO: 6) resulting in an amplified fragment of 729 bp. The 729 bp fragment was purified and restriction digested with BamHI and XhoI. The resulting 717 bp fragment was cloned into the corresponding restriction endonuclease sites of pJaL790 giving an Aspergillus expression plasmid named pNZ10. The complete amino acid sequence of the heterologous signal fused to the light-chain is given SEQ ID NO: 25.

### EXAMPLE 10

Construction of the hemA Minus A. oryzae Strain, ICA133

For removing the defect pyrG gene resident in the alkaline protease gene in the A. oryzae strain BECh2 the following was done:

A. Isolation of a pyrGminus A. oryzae strain, ToC1418

The A. oryzae strain BECh2 was screened for resistance to 5-flouro-orotic acid (FOA) to identify spontaneous pyrG mutants on minimal plates (Cove D. J., 1966, Biochem. Biophys. Acta. 113:51-56) supplemented with 1.0 M sucrose as carbon source, 10 mM sodiumnitrate as nitrogen source, and 0.5 mg/ml FOA. One strain, ToC1418, was identifying as being pyrGminus. ToC1418 is uridine dependent, therefore it can be transformed with the wild type pyrG gene and transformants selected by the ability to grow in the absence of uridine.

B. Construction of a pyrGplus A. oryzae Strain, JaL352

The mutation in the defect pyrG gene resident in the alkaline protease gene was determined by sequencing. Chromosomal DNA from A. oryzae strain BECh2 was prepared and by PCR with primers 104025 (SEQ ID NO: 31) and 104026 (SEQ ID NO: 32) a 933 bp fragment was amplified containing the coding region of the defect pyrG gene. The 933 bp fragment was purified and sequenced with the following primers: 104025, 104026, 104027 (SEQ ID NO: 33), 104028 (SEQ ID NO: 34), 108089 (SEQ ID NO: 35), and 108091 (SEQ ID NO: 36). Sequencing shows that an extra base, a G, was inserted at position 514 in the pyrG-coding region (counting from the A in the start codon of the pyrG gene), thereby creating a frame-shift mutation.

To make a wild type pyrG gene out of the defect pyrG gene resident in the alkaline protease the A. oryzae pyrGminus strain ToC1418 was transformed with 150 pmol of an oligo-nucleotide (SEQ ID NO: 37) phosphorylerated in the 5′ end, using standard procedures. The oligo-nucleotide restores the pyrG reading frame, but at the same time a silence mutation is introduce thereby creating a StuI restriction endonuclease site. Transformants were then selected by their ability to grow in the absence of uridine on minimal plates (Cove D. J., 1966, Biochem. Biophys. Acta. 113:51-56) supplemented with 1.0 M sucrose as carbon source, and 10 mM sodiumnitrate as nitrogen source. After reisolation chromosomal DNA was prepared from 8 transformants. To confirm the changes a 785 bp fragment was amplified by PCR with the primers 135944 (SEQ ID NO: 38) and 108089 (SEQ ID NO: 35), which is covering the region of interest. The 785 bp fragment was purified and sequenced with the primers 108089 (SEQ ID NO: 35) and 135944 (SEQ ID NO: 38). One strain having the expected changes was named JaL352.

C. Isolation of a pyrGminus A. oryzae strain, JaL355

For removing the pyrG gene resident in the alkaline protease gene JaL352 was transformed by standard procedure with the 5.6 kb BamHI fragment of pJaL173 harboring the 5′ and 3′ flanking sequence of the A. oryzae alkaline protease gene. Protoplasts were regenerated on non-selective plates and spores were collected. About 109 spores were screened for resistance to FOA to identify pyrG mutants. After reisolation chromosomal DNA was prepared from 14 FOA resistance transformants. The chromosomal DNA was digested with Bal I and analyzed by Southern blotting, using the 1 kb 32P-labelled DNA Bal I fragment from pJaL173 containing part of the 5′ and 3′ flanks of the A. oryzae alkaline protease gene as the probe. Strains of interest were identified by the disappearance of a 4.8 kb Bal I band and the appearance of a 1 kb Bal I band. Probing the same filter with the 3.5 kb 32P-labelled DNA Hind III fragment from pJaL335 containing the A. oryzae pyrG gene gives that the 4.8 kb Bal I band is disappeared in the strains of interest. One strain resulting from these transformants was named JaL355.

D. Construction of a hemA Minus A. oryzae Strain, ICA133

From the A. oryzae hemA gene sequence given in U.S. Pat. No. 6,033,892 (Genbank:AF152374), primers were designed to amplify the 5′ flanking and the 3′ flanking sequences. The primers for the 5′ flanking part, B2340E06 (SEQ ID NO: 39) and B2340E07 (SEQ ID NO: 40) were tailed with BspLU11I and Xho I sites, respectively. The primers for the 3′ flanking part B2340E08 (SEQ ID NO. 41) and B2340E09 (SEQ ID NO: 42) were tailed with Xho I and Not I sites, respectively.

The amplified fragments on 1068 bp and 1153 bp were digested with BspLU11I-Xho I and Xho I-Not I, resulting in 1049 bp fragment and 1132 bp fragment, respectively. These fragments were then cloned into BspLU11I-Not I digested pDV8 (vector for positive negative selection). Finally, the pyrG gene of A. oryzae flanked by direct repeats were isolated as a 2346 bp Sal I fragment of pJaL554 and inserted into the Xho I site formed between the 5′ and 3′ flanking fragment. The formed plasmid was termed pICA128.

pICA128 was linearized with Not I and used to transform A. oryzae JaL355 and transformants were selected on minimal medium plates supplemented with 250 mM 5′-aminolevulinic acid (5-ALA) and 0.6 mM 5-fluoro-2′-deoxyuridine (FdU) as described in WO 01/68864. A number of transformants were reisolated and plated onto Cove plates without 5-ALA. Two transformants (#2 and #7) growing well on Cove supplemented with 5-ALA, but not growing on Cove without 5-ALA were selected for Southern blot analysis. The chromosomal DNA was digested with Bgl II and analyzed by Southern blotting, using the 1049 bp 32P-labelled DNA BspLU11I-Xho I fragment from pICA128 containing part of the 5′ flanks of the A. oryzae hemA gene as the probe. Strains of interest were identified by the disappearance of a 1.8 kb Bgl II band and the appearance of a 7.5 kb Bgl II band. The filter was stripped and reprobed with a 476 bp 32P-labelled DNA Sal I-Pst I internal fragment of the A. oryzae hemA encoding part. No hybridization signals are expected if pICA128 integrates by homologous double cross over. For both transformants no hybridization signal was seen. One of the transformants was named ICA133.

### EXAMPLE 11

Construction of the pyrG Minus A. oryzae Strain ToC1512

A. Construction of a glaA Minus A. oryzae strain, ToC1510

Out from the A. oryzae IFO 4177 glycoamylase A (glaA) gene sequence (SEQ ID NO: 43), primers were designed to amplify the 5′ flanking and the 3′ flanking sequences. The primers for the 5′ flanking part, 101687 (SEQ ID NO: 44) and 101688 (SEQ ID NO: 45) were tailed with Bgl II and Hind III sites, respectively. The primers for the 3′ flanking part 101689 (SEQ ID NO: 46) and 101690 (SEQ ID NO: 47) were tailed with Hind III and Sal I sites, respectively.

The amplified fragments on 1073 bp and 1049 bp were digested with Bgl II-Hind III and Hind III- Sal I, resulting in a 1061 bp fragment and a 1037 bp fragment, respectively. These two fragments were then cloned into Bgl II-Sal I digested pUC19R, resulting in plasmid pToC381. The 2104 bp BamHI-Bgl II fragment from pToC381 was blunt ended by treatment with Klenow and the four deoxyribonucleotides and cloned into pDV8 digested with Hind III and blunt ended by treatment with Klenow and the four deoxyribonucleotides giving plasmid pToC465. Finally, the pyrG gene of A. oryzae flanked by direct repeats was isolated as a 2545 bp Hind III fragment of pJaL554 and inserted into the Hind III site formed between the 5′ and 3′ flanking fragment. The formed plasmid was termed pToC466.

pToC466 was linearized with Not I and used to transform A. oryzae JaL355 and transformants were selected on minimal medium 0.6 mM 5-fluoro-2′-deoxyuridine (FdU) as described in WO 0168864. A number of transformants were reisolated twice and genomic DNA was prepared. The chromosomal DNA from each of the transformants was digested with Pvu I and analyzed by Southern blofting, using the 1061 bp 32P-labelled DNA Hind III-Bgl II fragment from pToC381 containing the 5′ flanks of the A. oryzae glaA gene as the probe. Strains of in-terest were identified by the disappearance of a 1.6 kb Pvu I band and the appearance of a 7.3 kb Pvu I band. The filter was stripped and reprobed with a 1020 bp 32P-labelled DNA PCR fragment amplified from A. oryzae genomic DNA by the primers: 101691 (SEQ ID NO: 48) and 101692 (SEQ ID NO: 49). This PCR fragment encoded part of the A. oryzae glaA protein. No hybridization signals are expected if pToC466 integrates by homologous double cross over, where in JaL355 there is a 1.6 kb band. One transformant having the above characteristics was named ToC1510.

B. Isolation of a pyrG Minus A. oryzae Strain, ToC1512

The A. oryzae strain ToC1510 was screened for resistance to 5-flouro-orotic acid (FOA) to identify spontaneous pyrG mutants on minimal plates (Cove D. J., 1966, Biochem. Biophys. Acta. 113:51-56) supplemented with 1.0 M sucrose as carbon source, 10 mM sodiumnitrate as nitrogen source, and 0.5 mg/ml FOA. One strain, ToC112, was identifying as being pyrGminus. ToC1512 is uridine dependent, therefore it can be transformed with the wild type pyrG gene and transformants selected by the ability to grow in the absence of uridine.

### EXAMPLE 12

Expression of IgG1 Heavy Chain in Aspergillus oryzae

The strain ICA133 was transformed with either of the expression plasmid pNZ-3, -5, -7 and -9 as described by Christensen et al.; Biotechnology 1988 6 1419-1422. In short, A. oryzae mycelia were grown in a rich nutrient broth. The mycelia were separated from the broth by filtration. The enzyme preparation Novozyme® (Novo Nordisk) was added to the mycelia in osmotically stabilizing buffer such as 1.2 M MgSO₄ buffered to pH 5.0 with sodium phosphate. The suspension was incubated for 60 minutes at 37° C. with agitation. The protoplast was filtered through mira-cloth to remove mycelial debris. The protoplast was harvested and washed twice with STC (1.2 M sorbitol, 10 mM CaCl₂, 10 mM Tris-HCl pH 7.5). The protoplasts were finally resuspended in 200-1000 microl STC.

For transformation 5 microg DNA was added to 100 microl protoplast suspension and then 200 microl PEG solution (60% PEG 4000, 10 mM CaCl₂, 10 mM Tris-HCl pH 7.5) was added and the mixture was incubated for 20 minutes at room temperature. The protoplast were harvested and washed twice with 1.2 M sorbitol. The protoplast was finally resuspended 200 microl 1.2 M sorbitol. Transformants containing the amdS gene were selected on minimal plates (Cove D. J., 1966, Biochem. Biophys. Acta. 113:51-56) containing 1.0 M sucrose as carbon source, 10 mM acetamide as nitrogen source, 15 mM CsCl to inhibit background growth, and 250 mM 5-ALA. After 4-5 days of growth at 37° C., stable transformants appeared as vigorously growing and sporulating colonies. Transformants were purified twice through conidiospores.

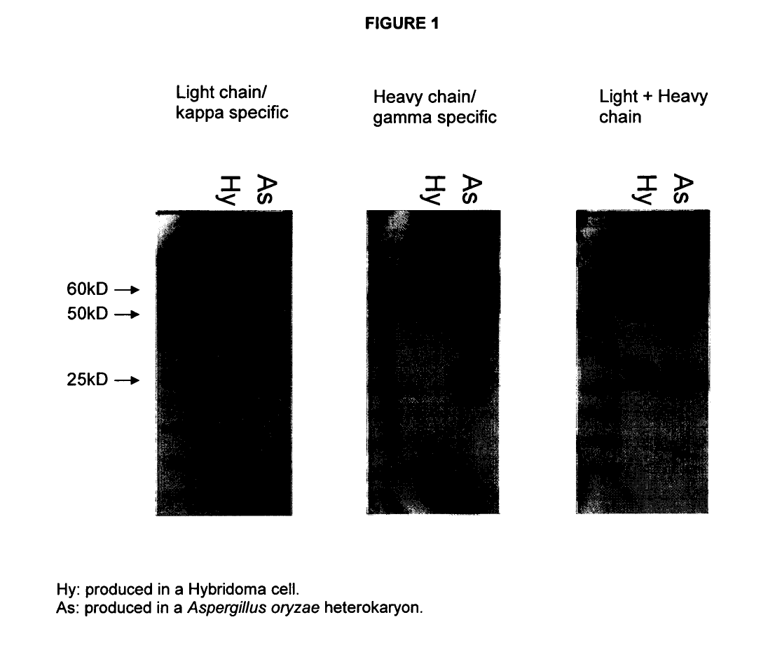

Shake flask containing 10 ml YPM medium (2 g/l yeast extract, 2 g/l peptone, and 2% maltose) was inoculated with spores from the transformants and incubated at 30° C., 200 rpm for 4 days. Samples of supernatant (20 microl) were mixed with an appropriate volume of 2× sample loading buffer and subjected to sodium dodecyl sulfate polyacrylamide gel electrophoresis (SDS-PAGE) according to the manufacturer's instructions (Novex NuPAGE 10% Bis-Tris Electrophoresis System from Invitrogen Corporation). The gels were stained for protein with Coomassie Brilliant Blue stain or the protein was transferred to membrane filters by Western blotting (Towbin et al., 1979, Proc. Natl. Acad. Sci. USA 76:43504354). The gels were run with a standard protein marker and supernatant from hybridoma cells expressing the same human heavy chain as the A. oryzae cell. Human heavy chain was detected on Western blots by treatment with anti-human IgG (gamma-chain specific) conjugated with alkaline phosphatase (AP) from goat (Sigma A3187) followed by AP color development by incubation with 4-nitro-phenyl phosphate (Sigma N7653) according to the manufacturer's instructions. A Western blot of the heavy chain is shown in FIG. 1, second gel.

Transformants which produced the heavy chain were identified by the appearance of an extra band of 55-60 kD compared to supernatant from an untransformed parental strain.

### EXAMPLE 13

Expression of a kappa Light Chain in Aspergillus oryzae

The strain ToC1512 was transformed with either of the expression plasmid pNZ-4, -6, -8 and -10 as described for the heavy chain in example 12, with the exception that the 250 mM 5-ALA was substituted with 20 mM uridine.

Shake flask containing 10 ml YPM medium (2 g/l yeast extract, 2 g/l peptone, and 2% maltose) was inoculated with spores from the transformants and incubated at 30° C., 200 rpm for 4 days. Samples of supernatant (20 microl) were mixed with an appropriate volume of 2× sample loading buffer and subjected to sodium dodecyl sulfate polyacrylamide gel electrophoresis (SDS-PAGE) according to the manufacturer's instructions (Novex NuPAGE 10% Bis-Tris Electrophoresis System from Invitrogen Corporation). The gels were stained for protein with Coomassie Brilliant Blue stain or the protein was transferred to membrane filters by Western blotting (Towbin et al., 1979, Proc. Natl. Acad. Sci. USA 76:43504354). The gels were run with a standard protein marker and supernatant from hybridoma cells expressing the same human kappa light chain as the A. oryzae cell. Human kappa light chain was detected on Western blots by treatment with anti-human kappa light chain antibody conjugated with alkaline phosphatase (AP) from goat (Sigma A3813) followed by AP color development by incubation with 4-nitro-phenyl phosphate (Sigma N7653) according to the manufacturer's instructions. A Western blot of the light chain is shown in FIG. 1, the first gel.

Transformants which produced the light chain were identified by the appearance of an extra band on 25 kD compared to supernatant from an untransformed parental strain. The identities of the light chain bands were further confirmed by determinations of the N-terminal end by Edman degradation. These data shows that a single dominant sequence of DIQMTQS (SEQ ID NO: 51) was obtained for all 4 expression constructs, which corresponds to the human isolated antibody analog.

### EXAMPLE 14

Expression of Intact IgG1 Antibody in Aspergillus oryzae Heterokaryons

The formation of Aspergillus oryzae heterokaryon cells having mixed nuclei encoding the kappa light chain and the IgG1 heavy chain was done as followed: Approximately 10⁵ spores of a transformant expressing a heavy chain from example 12, which are hemA negative, and of a transformant expressing a light chain from example 13, which are pyrG negative, were mixed in 15 ml COVE media (Cove D. J., 1966, Biochem. Biophys. Acta. 113:51-56) supplemented with 0.02 mM uridine and 25 mM 5′-ALA in a 25 ml NUNC universal container (NUNC 364228). This was incubated for 2 days at 30° C. without shaking. The surface mycelia mats were washed 2 times in sterile water, transferred to COVE plates and incubated 3 days at 37° C. A 1.0 cm square agar plug was transferred to a new COVE plate and incubated 3 days at 37° C. All subsequent handlings of the heterokaryons were done on/in media selecting for heterokaryons.

Shake flask containing 10 ml YPM medium (2 g/l yeast extract, 2 g/l peptone, and 2% maltose) was inoculated with spores from the heterokaryons and incubated at 30° C., 200 rpm for 4 days. The supernatants were analyzed for expression of heavy and light chain samples by SDS-page and Western analysis. The gels were run with a standard protein marker and supernatant from hybridoma cells expressing the same human kappa light chain and heavy chain as the A. oryzae heterokaryon.

From one heterokaryon having the native heavy chain (from example 12) and the CBD light chain fusion (from example 13), the heavy chain associated with the light chain was obtained by protein A chromatography (Goudswaard et al., 1978, Scand J Immunol, 8: 21-28), which is specific for heavy chain. FIG. 1, third gel shows the results of a Western blot using the antibodies described in example 12 and 13 that are specific for the heavy and light chain. The bands observed for the transformant were identified as the heavy chain (50, 53 and 55 kD, probably different glycol forms) and the light chain (25 kD). That the light chain was co-purified with the heavy chain demonstrated that the antibody was assembled.

N-terminal end determination of the bands confirmed that the 3 heavy chain bands had the same sequence, namely EGQLVQSG (SEQ ID NO: 52) and the light chain had the sequence of DIQMTQS (SEQ ID NO: 51), which for both the heavy and light chain corresponds to the sequence of the antibody produced by hybridoma cells.

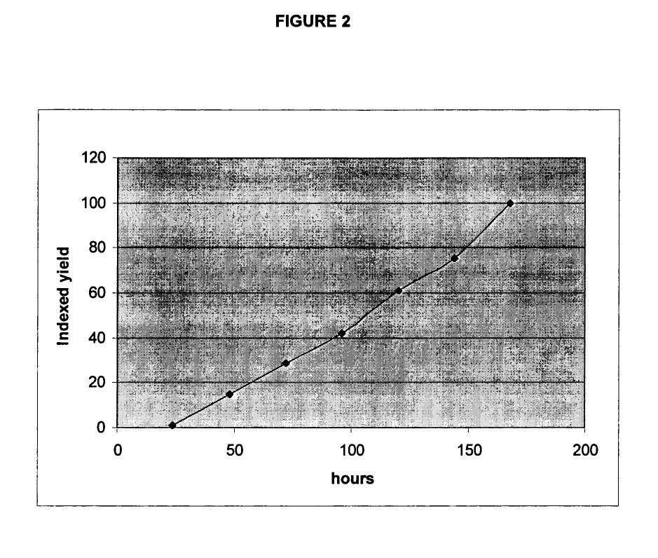

Furthermore, the amount of functional antibody expressed by the A. oryzae heterokaryon was measured in a 3 liter-tank and is shown in FIG. 2 and in below table 1 as a function of how many hours the heterokaryon has been cultured. The yield is given relative to the amount produced after 168 hours and it was determined by the binding to the antigen of the antibody.

TABLE 1

| Hours | Relative yield |
|---|---|
| 24 | 1 |
| 48 | 15 |
| 72 | 29 |
| 96 | 42 |
| 120 | 61 |
| 144 | 75 |
| 168 | 100 |

### EXAMPLE 15

The Effect of Different Culture Conditions on the Amount of Antibody Expression

The A. oryzae from example 14 was used in the present example to test the effect of different culture conditions on the amount of antibody expressed by the heterokaryon. 500-ml shake-flasks containing 200 ml G2Gly medium (18 g/L yeast extract and 24 g/L glycerol) were inoculated with 10 mL of spore suspension. After 24-48 hrs of growth at 30° C. in a shaking-incubator, they were transferred to 3L Applicon Bioreactors. Medium and fermentation parameters for the J3 culture were as follows:

Fermentation medium in the Bioreactor: 60 g Sucrose, 12 g (NH₄)₂SO₄, 8 g MgSO₄·7 H₂O, 8 g KH₂PO₄, 1.0 mL, Tracemetal solution, 3.4 mL Pluronic (antifoam) per 2.0 L, this was autoclaved at 121° C. for 60 min and the pH was adjusted to 6.5.

After about 14-18 hours of culture (batch-phase) the culture is fed by continuously adding a so-called feed-medium. The feed-medium consisted in the present case of 3.5 g citric acid, 20 mL pluronic (antifoam) and 2 L Nutriose (maltose), BRIX40 (this gives a concentration of 40% maltose-syrup in the feed medium as determined by refractive index) in a total volume of 2.0 L and it had been autoclaved at 121° C. for 30 min before use.

The control parameters were as follows:

| | |
|---|---|
| pH: according to the set point table | Dosage: according to the set |
| | point table |
| Temperature: according to the set point | Base: 10% (w/w) NH₄OH |
| table | (approximately 0.5 L) |
| Stirring: Minimum 800 rpm and maximum | Air flow: 2.0 L/min |
| 1100 rpm | |
| Dissolved oxygen tension was controlled | Dosage starts when |
| to minimum 30% by the stirring | rpm(max) − rpm = 100 |

Set point table:

| pH | | Feed rate (g/min/L) | | Temperature | |
|---|---|---|---|---|---|
| | Set | | Set | | Set |
| Time | point | Time | point | Time | point |
| 0 | 6.5 | Feed start | 0.25 | 0 | 34 |
| Feed start | 7.0 | Feed start + | 0.15 | Feed start | 34 |
| | | 12 hrs | | | |
| Feed start + | 7.0 | Feed start + | 0.05 | Feed start + | 26 |
| 500 hrs | | 24 hrs | | 150 hrs | |

Time=0 is the start point of the fermentation, i.e. where the culture is transferred from the shaking-incubator to the 3L Applicon Bioreactors. Time=feed start is the start point for the addition of the feed-medium, which begins as described above (dosage start) when rpm(max)−rpm=100.

There is a linear change over time in the parameter value between two set points.

Twice a day a sample of the supernatant was withdrawn and the weight, pH and amount of glucose were determined. Furthermore, samples were withdrawn daily and supernatants were frozen awaiting antibody quantification.

The B2, C3, E2 and G1 fermentation experiments were performed similarly, with the exceptions/changes described in Table 2.

The amount of intact human IgG expressed was measured by ELISA as described in the “Methods” section and the results are shown in Table 2.

TABLE 2

| | | | | Amount of |
|---|---|---|---|---|
| | | | | expressed |
| | | | | antibody |
| Fermentation | | Maltose profile | Sucrose | (relative to |
| number | Temperature-profile | (g/min/L) | (g/L) | B2) |
| B2 | 34° C. | 0.05 | 25 | 1 |
| C3 | 34° C. during the | 0.06 | 30 | 1.19 |
| | fermentation, from | | | |
| | start of feed the | | | |
| | temperature was | | | |
| | lowered to 26° C. | | | |
| | over a period of 4 | | | |
| | hrs. | | | |
| E2 | 34° C. during the | Varied, | 30 | 1.07 |
| | fermentation, from | according to | | |
| | start of feed the | oxygen tension | | |
| | temperature was | | | |
| | lowered to 26° C. | | | |
| | over a period of 12 | | | |
| | hrs. | | | |
| J3 | 34° C. during the | 0.25 for 12 | 30 | 4.44 |
| | fermentation, from | hours, 0.15 | | |
| | start of feed the | between 12 and | | |
| | temperature was | 24 hours, 0.05 | | |
| | lowered to 26° C. | from 24 hours | | |
| | over a period of 150 | to the end | | |
| | hrs. | | | |

A temperature of 34° C. was initially used during the whole fermentation, mainly due to better growth of Aspergillus at this temperature (B2). A slight increased antibody concentration was obtained by changing the temperature to 26° C. over a period of 4 hours after the start of the feed (C3). In fermentation E2 the feed was controlled by the dissolved oxygen tension in the fermentor, which resulted in a higher biomass concentration, but it did not improve the final antibody concentration.

The antibody concentration increased more than fourfold when the temperature was linearly changed from 34° C. to 26° C. over 150 hrs from the beginning of the feed period (J3). The feed rate was controlled to imitate the one in fermentation E2, but with preset values which made it practically easier to control.

Hence the results in Table 2 indicate that reducing the temperature during the feed period increases the amount of antibody produced.

### EXAMPLE 16

Expression of Intact Human IgG in A. oryzae Heterokaryon from Different Combinations of Heavy and Light Chain Constructs

Example 14 shows expression of a human antibody by an A. oryzae heterokaryon comprising a construct of the wild-type heavy chain (corresponding to pNZ-3 from example 2) and the CBD light chain fusion protein (corresponding to pNZ-6—from example 5).

A. oryzae heterokaryons comprising different combinations of heavy and light chain constructs were formed as described in example 14.

The amount of intact human IgG expressed by the A. oryzae heterokaryons were measured by ELISA as described above in the “Methods” section and the results are shown in Table 3. The amount was normalised to the amount expressed by an A. oryzae heterokaryon comprising a construct of the wild-type heavy chain and a construct of the wild-type light chain (corresponding to pNZ-3 and pNZ-4 from examples 2 and 3, respectively).

TABLE 3

| Construct | | Relative |
|---|---|---|
| Heavy chain | Light chain | yield |
| Wild-type (pNZ-3) | Wild-type (pNZ-4) | 1 |
| Wild-type (pNZ-3) | CBD-fusion (pNZ-6) | 3.13 |
| CBD-fusion (pNZ-5) | Wild-type (pNZ-4) | 0.62 |
| CBD-fusion (pNZ-5) | CBD-fusion (pNZ-6) | 2.2 |
| Wild-type (pNZ-3) | TAKA-signal peptide (pNZ10) | 3.34 |
| CBD-fusion (pNZ-5) | TAKA-signal peptide (pNZ10) | 3.67 |
| Prepro from lipase B (pNZ-7) | CBD-fusion (pNZ-6) | 3.17 |
| Prepro from lipase B (pNZ-7) | TAKA-signal peptide (pNZ10) | 3.13 |

The results indicate that the A. oryzae heterokaryon is able to express the intact human IgG using different constructs and different combinations thereof, of the heavy and light chain.

### EXAMPLE 17

Construction of an IgG2 Heavy-chain CBD Fusion Aspergillus Expression Vector

A fusion protein between a human IgG2 heavy chain (SEQ ID NO: 55) and a cellulose binding domain from the endoglucanase II from M. giganteus was constructed by exchanging the DNA sequence encoding the native signal peptide of the heavy chain with the DNA sequence encoding the M. giganteus cellulose binding domain having its own signal and a linker ending with amino acids KR (CBD) by sequence overlap extension (SOE). The CBD was amplified by PCR using pA2C315 as template and the following pair of primers: the forward primer C315-N (SEQ ID NO: 7) and the reverse primer K1796F02 (SEQ ID NO: 63). The resulting PCR product on 260 bp was purified.

The heavy-chain was amplified by PCR using SEQ ID NO: 55 as template and the following pair of primers: the forward primer K1796F03 (SEQ ID NO: 64) and the reverse primer K1795F08 (SEQ ID NO: 67). The resulting PCR product on 1376 bp was purified.

The two above PCR products were mixed and a standard SOE PCR was preformed with the following pair of primers C315-N (SEQ ID NO: 7) and K1795F08 (SEQ ID NO: 67) resulting in an amplified fragment of 1596 bp. The 1596 bp fragment was purified and cut with BamHI and XhoI. The resulting 1584 bp fragment was cloned into the corresponding restriction endonuclease sites of pJaL790 giving an Aspergillus expression plasmid named pNZa-1. The complete sequence of the CBD fused to the heavy-chain is given in SEQ ID NO: 57.

### EXAMPLE 18

Construction of kappa Light-chain CBD Fusion Aspergillus Expression Vector

A fusion protein between a human kappa light chain (SEQ ID NO: 56) and the cellulose binding domain from the endoglucanase II from M. giganteus was constructed by exchanging the DNA sequence encoding the native signal peptide of the light chain with the DNA sequence encoding the M. giganteus cellulose binding domain having its own signal and a linker ending with amino acids KR (CBD) by sequence overlap extension (SOE).

The CBD was amplified by PCR on pA2C315 using the following pair of primers: the forward primer C315-N (SEQ ID NO: 7) and the reverse primer K1796F06 (SEQ ID NO: 68). The resulting PCR product on 260 bp was purified.

The light-chain were amplified by PCR out from SEQ ID NO: 56 using the following pair of primers: the forward primer K1796F07 (SEQ ID NO: 69) and the reverse primer K1795F09 (SEQ ID NO: 72). The resulting PCR product on 677 bp was purified.

The two above PCR products were mixed and a standard SOE PCR was preformed with the following pair of primers C315-N and K1795F09 resulting in and amplified fragment of 897 bp. The 897 bp fragment was purified and cut with BamHI and XhoI. The resulting 885 bp fragment was cloned into the corresponding restriction endonuclease sites of pJaL790 giving an Aspergillus expression plasmid named pNZa-2. The complete sequence of the CBD fused to the light-chain is given in SEQ ID NO: 59.

### EXAMPLE 19

Construction of IgG2 Heavy-chain Aspergillus Expression Plasmid with the TAKA Signal Peptide

The signal peptide sequence from Aspergillus oryzae alpha-amylase (TAKA) was exchanged with the native signal peptide of the human IgG2 heavy-chain used in example 17 by sequence overlap extension (SOE). The TAKA signal was amplified by PCR on pTAKA17 using the following pair of primers: the forward primer TAKA-17-N (SEQ ID NO: 20) and the reverse primer K1796F04 (SEQ ID NO: 65). The resulting PCR product on 95 bp was purified.

The heavy-chain was amplified by PCR using SEQ ID NO: 55 as template and the following pair of primers: the forward primer K1796F05 (SEQ ID NO: 66) and the reverse primer K1795F08 (SEQ ID NO: 67). The resulting PCR product on 1375 bp was purified.

The two above PCR products were mixed and a standard SOE PCR was preformed with the following pair of primers TAKA-17-N (SEQ ID NO: 20) and K1795F08 (SEQ ID NO: 67) resulting in an amplified fragment of 1431 bp. The 1431 bp fragment was purified and cut with BamHI and XhoI. The resulting 1419 bp fragment was cloned into the corresponding restriction endonuclease sites of pJaL790 giving an Aspergillus expression plasmid named pNZa-3. The complete sequence of the TAKA signal fused to the heavy-chain is given in SEQ ID NO: 61.

### EXAMPLE 20

Construction of kappa Light-chain Aspergillus Expression Plasmid with the TAKA Signal Peptide

The signal peptide sequence of the human kappa light-chain used in example 18 was exchanged with the signal peptide sequence from A. oryzae alpha-amylase (TAKA) by sequence overlap extension (SOE).

The TAKA signal was amplified by PCR using pTAKA17 as template and the following pair of primers: the forward primer TAKA-17-N (SEQ ID NO: 20) and the reverse primer K1796F08 (SEQ ID NO: 70). The resulting PCR product on 95 bp was purified.

The light-chain was amplified by PCR from SEQ ID NO: 56 using the following pair of primers: the forward primer K1796F09 (SEQ ID NO: 71) and the reverse primer K1795F09 (SEQ ID NO: 72). The resulting PCR product on 576 bp was purified.

The two above PCR products were mixed and a standard SOE PCR was preformed with the following pair of primers TAKA-17-N (SEQ ID NO: 20) and K1795F09 (SEQ ID NO: 72) resulting in an amplified fragment of 732 bp. The 732 bp fragment was purified and restriction digested with BamHI and XhoI. The resulting 720 bp fragment was cloned into the corresponding restriction endonuclease sites of pJaL790 giving an Aspergillus expression plasmid named pNZa-4. The complete sequence of the heterologous signal fused to the light-chain is given SEQ ID NO: 62.

### EXAMPLE 21

Expression of IgG2 Heavy Chain in Aspergillus oryzae

The strain ICA133 (example 10) was transformed with either of the expression plasmid pNZa-1, and -3 as described by Christensen et al.; Biotechnology, 1988, 6: 1419-1422. In short, A. oryzae mycelia were grown in a rich nutrient broth. The mycelia were separated from the broth by filtration. The enzyme preparation Novozyme® (Novozymes) was added to the mycelia in an osmotically stabilizing buffer such as 1.2 M MgSO₄ buffered to pH 5.0 with sodium phosphate. The suspension was incubated for 60 minutes at 37° C. with agitation. The protoplast was filtered through mira-cloth to remove mycelial debris. The protoplast was harvested and washed twice with STC (1.2 M sorbitol, 10 mM CaCl₂, 10 mM Tris-HCl pH 7.5). The protoplasts were finally re-suspended in 200-1000 microl STC.

For transformation 5 microg DNA was added to 100 microl protoplast suspension and then 200 microl PEG solution (60% PEG 4000, 10 mM CaCl₂, 10 mM Tris-HCl pH 7.5) was added and the mixture is incubated for 20 minutes at room temperature. The protoplasts were harvested and washed twice with 1.2 M sorbitol. The protoplasts were finally re-suspended 200 microl 1.2 M sorbitol. Transformants containing the amdS gene were selected on minimal plates (Cove D. J., 1966, Biochem. Biophys. Acta. 113:51-56) containing 1.0 M sucrose as carbon source, 10 mM acetamide as nitrogen source, 15 mM CsCl to inhibit background growth, and 250 mM 5-ALA. After 4-5 days of growth at 37° C., stable transformants appeared as vigorously growing and sporulating colonies. Transformants were purified twice through conidiospores.

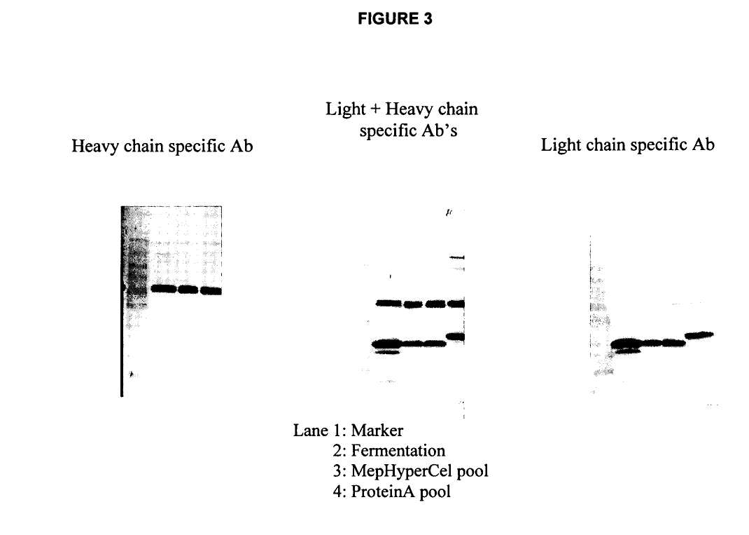

Shake flask containing 10 ml YPM medium (2 g/l yeast extract, 2 g/l peptone, and 2% maltose) was inoculated with spores from the transformants and incubated at 30° C., 200 rpm for 4 days. Samples of supernatant (20 microl) were mixed with an appropriate volume of 2× sample loading buffer and subjected to sodium dodecyl sulfate polyacrylamide gel electrophoresis (SDS-PAGE) according to the manufacturer's instructions (Novex NuPAGE 10% Bis-Tris Electrophoresis System from Invitrogen Corporation). The gels were stained for protein with Coomassie Brilliant Blue stain or the protein was transferred to membrane filters by Western blotting (Towbin et al., 1979, Proc. Natl. Acad. Sci. USA 76:43504354). Human heavy chain was detected on Western blots by treatment with anti-human IgG (gamma-chain specific) conjugated with alkaline phosphatase (AP) from goat (Sigma A3187) followed by AP color development by incubation with 4-nitro-phenyl phosphate (Sigma N7653) according to the manufacturer's instructions. A Western blot of the heavy chain is shown in FIG. 3, first gel.

Transformants which produced the heavy chain were identified by the appearance of an extra band of 55-60 kD compared to supernatant from an untransformed parental strain.

### EXAMPLE 22

Expression of a kappa Light Chain in Aspergillus oryzae

The strain ToC1512 (example 11) was transformed with either of the expression plasmid pNZa-2, and -4 as described for the heavy chain in example 21, with the exception that the 250 mM 5-ALA was substituted with 20 mM uridine.

Shake flask containing 10 ml YPM medium (2 g/l yeast extract, 2 g/l peptone, and 2% maltose) was inoculated with spores from the transformants and incubated at 30° C., 200 rpm for 4 days. Samples of supernatant (20 microl) were mixed with an appropriate volume of 2× sample loading buffer and subjected to sodium dodecyl sulfate polyacrylamide gel electrophoresis (SDS-PAGE) according to the manufacturer's instructions (Novex NuPAGE 10% Bis-Tris Electrophoresis System from Invitrogen Corporation). The gels were stained for protein with Coomassie Brilliant Blue stain or the protein was transferred to membrane filters by Western blotting (Towbin et al., 1979, Proc. Natl. Acad. Sci. USA 76:4350-4354). Human kappa light chain was detected on Western blots by treatment with anti-human kappa light chain antibody conjugated with alkaline phosphatase (AP) from goat (Sigma A3813) followed by AP color development by incubation with 4-nitro-phenyl phosphate (Sigma N7653) according to the manufacturer's instructions. A Western blot of the light chain is shown in FIG. 3, third gel.

Transformants which produced the light chain were identified by the appearance of an extra band on 25 kD compared to supernatant from an untransformed parental strain. The identities of the light chain bands were further confirmed by determinations of the N-terminal end by Edman degradation. These data shows that a single dominant sequence of EIVLTQS (SEQ ID NO: 73) was obtained for all 4 expression constructs, which corresponds to the human isolated antibody analog.

### EXAMPLE 23

Expression of Intact IgG2 Antibody in Aspergillus oryzae Heterokaryons

The formation of Aspergillus oryzae heterokaryon cells having mixed nuclei encoding the kappa light chain and the IgG2 heavy chain was done as followed: Approximately 10⁵ spores of a transformant expressing a heavy chain from example 21, which are hemA negative, and of a transformant expressing a light chain from example 22, which are pyrG negative, were mixed in 15 ml COVE media (Cove D. J., 1966, Biochem. Biophys. Acta. 113:51-56) supplemented with 0.02 mM uridine and 25 mM 5′-ALA in a 25 ml NUNC universal container (NUNC 364228). This was incubated for 2 days at 30° C. without shaking. The surface mycelia mats were washed 2 times in sterile water, transferred to COVE plates and incubated 3 days at 37° C. A 1.0 cm square agar plug was transferred to a new COVE plate and incubated 3 days at 37° C. All subsequent handlings of the heterokaryons were done on/in media selecting for heterokaryons.

Shake flask containing 10 ml YPM medium (2 g/l yeast extract, 2 g/l peptone, and 2% maltose) was inoculated with spores from the heterokaryons and incubated at 30° C., 200 rpm for 4 days. The supernatants were analyzed for expression of heavy and light chain samples by SDS-page and Western analysis.

From one heterokaryon having the TAKA signal heavy chain (from example 21) and the TAKA light chain fusion (from example 22), the heavy chain associated with the light chain was obtained by protein A chromatography (Goudswaard et al., 1978, Scand, J. Immunol., 8: 21-28), which is specific for heavy chain. FIG. 3 shows gels of the results of a Western blot using the antibodies described in example 21 and 22 that are specific for the heavy and light chain. The bands observed for the transformant were identified as the heavy chain (50, and 55 kD, probably different glycol forms) and the light chain (25 kD). That the light chain was co-purified with the heavy chain demonstrated that the antibody was assembled.

N-terminal end determination of the bands confirmed that the 2 heavy chain bands had the same sequence, namely EVQLLQSG (SEQ ID NO: 74) and the light chain had the sequence of EIVLTQS (SEQ ID NO: 73), which for both the heavy and light chain corresponds to the sequence of the antibody produced by CHO cells.

### EXAMPLE 24

Expression of Intact Human IgG in A. oryzae Heterokaryon from Different Combinations of Heavy and Light Chain Constructs

A. oryzae heterokaryons comprising constructs of both heavy and light CBD fusion protein (example 17 and 18) or constructs of both TAKA-signal peptide with the heavy and light chain (example 19 and 20) were formed as described in example 23.

The amount of intact human IgG2 expressed by the A. oryzae heterokaryons were measured by ELISA as described above in the “Methods” section and showed that the heterokaryons comprising the TAKA-signal peptide on both the heavy and light chain expressed approximately 2.6 times as much intact antibody as those comprising CBD heavy and light chain fusions.

1. A method for producing a monoclonal antibody comprising
(a) providing a heterokaryon fungus comprising a first nucleus and a second nucleus, wherein said first nucleus comprises a first nucleic acid construct comprising a first nucleic acid sequence encoding a light chain of an antibody, and wherein said second nucleus comprises a second nucleic acid construct comprising a first nucleic acid sequence encoding a heavy chain of an antibody, and wherein at least one of said nucleic acid constructs further comprises a second nucleic acid sequence encoding a polypeptide or a functional part thereof normally secreted by a fungus
(b) culturing the heterokaryon fungus under conditions suitable for expression of the antibody light and heavy chains.

2. A method of claim 1, wherein the heterokaryon is a filamentous heterokaryon fungus.

3. A method of claim 1, wherein the heterokaryon is a yeast heterokaryon.

4. A method of claim 1, wherein the second nucleic acid sequence is located upstream of the first nucleic acid sequence.

5. A method of claim 1, wherein the second nucleic acid sequence encodes a cellulose binding domain.

6. A method of claim 1, wherein at least one of said nucleic acid constructs further comprises a third nucleic acid sequence encoding a signal peptide, and wherein said signal peptide is heterologous to the first nucleic acid sequence of said nucleic acid construct.

7. A method of claim 6, wherein the signal peptide is derived from the alpha-amylase gene from Aspergillus oryzae (SEQ ID NO: 26).

8. A method of claim 6, wherein the signal peptide is derived from the lipase gene from Candida antarctica (SEQ ID NO: 27).

9. A method of claim 8, wherein at least one of said nucleic acid constructs further comprises a nucleic acid sequence encoding the pro-sequence derived from the lipase B gene from Candida antarctica (SEQ ID NO: 28).

10. A method of claim 1, wherein either expression of the light chain from the first nucleic acid construct or expression of the heavy chain from the second nucleic acid construct or both is/are under the control of an inducible promoter, and wherein the culturing in step (b) is performed by first culturing the heterokaryon fungus under conditions where the promoter is not induced and subsequently under conditions where the promoter is induced, and wherein the temperature is lowered during culturing under conditions where the promoter is induced.

11. A method for producing a monoclonal antibody comprising
(a) providing a heterokaryon fungus comprising a first nucleus and a second nucleus, wherein said first nucleus comprises a first nucleic acid construct comprising a first nucleic acid sequence encoding a light chain of an antibody, and wherein said second nucleus comprises a second nucleic acid construct comprising a first nucleic acid sequence encoding a heavy chain of an antibody, and wherein at least one of said nucleic acid constructs further comprises a third nucleic acid sequence encoding a signal peptide heterologous to the first nucleic acid sequence
(b) culturing the heterokaryon fungus under conditions suitable for expression of the antibody light and heavy chains.

12. A method of claim 11, wherein the heterokaryon is a filamentous heterokaryon fungus.

13. A method of claim 11, wherein the heterokaryon is a yeast heterokaryon.

14. A method of claim 11, wherein the signal peptide is derived from the alpha-amylase gene from Aspergillus oryzae (SEQ ID NO: 26).

15. A method of claim 11, wherein the signal peptide is derived from the lipase gene from Candida antarctica (SEQ ID NO: 27).

16. A method of claim 15, wherein at least one of said nucleic acid constructs further comprises a nucleic acid sequence encoding the pro-sequence derived from the lipase B gene from Candida antarctica (SEQ ID NO: 28).

17. A method of claim 11, wherein either expression of the light chain from the first nucleic acid construct or expression of the heavy chain from the second nucleic acid construct or both is/are under the control of an inducible promoter, and wherein the culturing in step b) is performed by first culturing the heterokaryon fungus under conditions where the promoter is not induced and subsequently under conditions where the promoter is induced, and wherein the temperature is lowered during culturing under conditions where the promoter is induced.

18-27. (canceled)
